SEQUENCE LISTING (40 sequences)

SEQ ID NO: 1 - protein, 119 aa, Homo sapiens
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSS

SEQ ID NO: 2 - protein, 107 aa, Homo sapiens
DIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIK

SEQ ID NO: 3 - protein, 5 aa, Homo sapiens
RYWMS

SEQ ID NO: 4 - protein, 17 aa, Homo sapiens
EINPDSSTINYTPSLKD

SEQ ID NO: 5 - protein, 10 aa, Homo sapiens
PDGNYWYFDV

SEQ ID NO: 6 - protein, 11 aa, Homo sapiens
KASQDVGIAVA

SEQ ID NO: 7 - protein, 7 aa, Homo sapiens
WASTRHT

SEQ ID NO: 8 - protein, 9 aa, Homo sapiens
QQYSSYPYT

SEQ ID NO: 9 - protein, 119 aa, Homo sapiens
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 10 - protein, 107 aa, Homo sapiens
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIK

SEQ ID NO: 11 - protein, 5 aa, Homo sapiens
RYWMS

SEQ ID NO: 12 - protein, 17 aa, Homo sapiens
EINPDSSTINYAPSLKD

SEQ ID NO: 13 - protein, 10 aa, Homo sapiens
PDGNYWYFDV

SEQ ID NO: 14 - protein, 11 aa, Homo sapiens
KASQDVGIAVA

SEQ ID NO: 15 - protein, 7 aa, Homo sapiens
WASTRHT

SEQ ID NO: 16 - protein, 9 aa, Homo sapiens
QQYSSYPYT

SEQ ID NO: 17 - protein, 120 aa, Homo sapiens
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 18 - protein, 107 aa, Homo sapiens
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 19 - protein, 5 aa, Homo sapiens
TYWMN

SEQ ID NO: 20 - protein, 17 aa, Homo sapiens
MIHPSDSETRLNQKFKD

SEQ ID NO: 21 - protein, 11 aa, Homo sapiens
STMIATRAMDY

SEQ ID NO: 22 - protein, 11 aa, Homo sapiens
KASQDVITGVA

SEQ ID NO: 23 - protein, 7 aa, Homo sapiens
SASYRYT

SEQ ID NO: 24 - protein, 9 aa, Homo sapiens
QQHYSTPLT

SEQ ID NO: 25 - protein, 121 aa, Homo sapiens
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSA

SEQ ID NO: 26 - protein, 107 aa, Homo sapiens
DIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIK

SEQ ID NO: 27 - protein, 5 aa, Homo sapiens
SYWMQ

SEQ ID NO: 28 - protein, 17 aa, Homo sapiens
AIYPGDGDTRYTQKFKG

SEQ ID NO: 29 - protein, 12 aa, Homo sapiens
GKVYYGSNPFAY

SEQ ID NO: 30 - protein, 11 aa, Homo sapiens
KASDHINNWLA

SEQ ID NO: 31 - protein, 7 aa, Homo sapiens
GATSLET

SEQ ID NO: 32 - protein, 9 aa, Homo sapiens
QQYWSTPWT

SEQ ID NO: 33 - protein, 120 aa, Mus musculus
QVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSS

SEQ ID NO: 34 - protein, 108 aa, Mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIK

SEQ ID NO: 35 - protein, 5 aa, Mus musculus
SSWMN

SEQ ID NO: 36 - protein, 17 aa, Mus musculus
RIYPGDGDTKYNGKFKG

SEQ ID NO: 37 - protein, 11 aa, Mus musculus
STMIATGAMDY

SEQ ID NO: 38 - protein, 11 aa, Mus musculus
KASQDVSTAVA

SEQ ID NO: 39 - protein, 7 aa, Mus musculus
SASYRYT

SEQ ID NO: 40 - protein, 10 aa, Mus musculus
QQHYSTPPYT

(19) United States
(12) Patent Application Publication
(10) Pub. No.: US 2013/0058921 A1
(43) Pub. Date: Mar. 7, 2013

(54) USE OF AUTOLOGOUS EFFECTOR CELLS AND ANTIBODIES FOR TREATMENT OF MULTIPLE MYELOMA

(71) Applicant: Frits Van Rhee, Little Rock, AR (US)

(72) Inventor: Frits Van Rhee, Little Rock, AR (US)

(21) Appl. No.: 13/505,005

(22) PCT Filed: Oct. 30, 2009
(86) PCT No.: PCT/US09/62874

§ 371 Date: Nov. 14, 2012

(51) Int. Cl. A61K 39/395; A61P 35/00

(52) U.S. Cl. 424/133.1; 424/173.1; 424/178.1; 424/158.1

(57) ABSTRACT

The present disclosure provides methods for treating multiple myeloma using a combination of autologous expanded and activated NK cells from the patient and an antibody that targets an antigen on myeloma cells and/or an antibody that targets the KIR antigen on NK cells, wherein the antibodies elicit ADCC toward myeloma cells. The present disclosure provides methods for treating multiple myeloma using a combination of autologous NK cells and the anti-CS1 antibody elotuzumab.

### 1. BACKGROUND

Multiple myeloma (also referred to herein as “myeloma”) is a malignant proliferation of plasma cells that produce monoclonal immunoglobulin. Multiple myeloma cells also express on their surface the protein CS1 (also known as SLAMF7, CRACC, 19A, APEX-I and FOAP12), a member of the CD2 family of cell surface glycoproteins that is not expressed on normal tissues or on CD34⁺ stem cells. The myeloma tumor, its products, and the host response to it result in symptoms including persistent bone pain or fracture, renal failure, susceptibility to infection, anemia, hypercalcemia, and occasionally clotting abnormalities, neurologic symptoms and vascular manifestations of hyperviscosity. (See D. Longo, in Harrison's Principles of Internal Medicine 14th Edition 713 (McGraw-Hill, New York, 1998)). Multiple myeloma is a progressive and incurable disease that affects 14,400 new individuals in the United States annually (See Anderson et al. (1999) Introduction. Seminars in Oncology 26:1).

Multiple myeloma is difficult to diagnose early because there may be no symptoms in the early stages. Furthermore, no effective long-term treatment currently exists for the disease. The median duration of survival is six months when no treatment is given. The main treatment for multiple myeloma is systemic chemotherapy with agents such as melphalan, thalidomide, cyclophosphamide, doxorubicin, lenalidomide (Revlimid®) or bortezomib (Velcade®), either alone or in combination. However, some patients do not respond to chemotherapy. The current median of survival is greater than 5 years as a result of advances in treatment. Nevertheless, fewer than 5% of patients live longer than 10 years (See Anderson et al. (1999) Annual Meeting Report 1999. Recent Advances in the Biology and Treatment of Multiple Myeloma).

Additional treatment strategies include high-dose therapy with autologous hematopoietic cell transplantation (HCT), tandem autografts, and high-dose conditioning with allogeneic HCT. Allogeneic HCT is associated with a higher frequency of sustained remissions and a lower risk of relapse due to the graft-versus-tumor activity resulting from immune response to minor antigen differences between donor and host. Unfortunately, allogeneic HCT is also associated with high transplantation-related mortality, due in part to graft versus host disease (GVHD). Approaches using nonmyeloablative conditioning and novel posttransplantation immunosuppression to assure engraftment and graft-versus-tumor effects have reduced the transplantation related mortality. (See, e.g., Maloney, et al. (2003) Blood 102:3447).

Recently, killer immunoglobulin-like receptor-ligand mismatched natural killer (“NK”) cell transfusions from haplo-identical donors achieved near complete remission in 50% of multiple myeloma patients in the trial. (Shi et al. (2008) Brit. J. Haemotol. 143:641). Nevertheless, 2 out of the 10 patients in this study had progressive disease, and the median duration of response was only 105 days for the other 8 patients.

There is a need for additional multiple myeloma therapies that do not rely on the availability of appropriate donors, that effectively kill myeloma cells without killing normal cells, and that do not elicit early rejection in patients.

### 2. SUMMARY

Multiple myeloma is a progressive and at present incurable cancer of the plasma cells. Current therapies are aimed at the amelioration of myeloma symptoms and long term survival. CS1 (CRACC, SLAMF7, CD319), a member of the signaling lymphocyte activating molecule-related receptor family, is highly expressed on myeloma cells. Other proteins which are expressed on myeloma cells include, but are not limited to, CD20, CD38, CD40, CD56, CD74, CD138, CD317 (also known as HM1.24 antigen), IGF receptor, IL6 receptor, TRAIL receptor 1 and TRAIL receptor 2. Targeting CS1 in myeloma cells has been shown to inhibit the proliferation of cancer cells. For example, the anti-CS1 antibody elotuzumab (HuLuc63) exhibits in vitro antibody-dependent cellular cytotoxicity (ADCC) in primary myeloma cells and in vivo anti-tumor activity (Hsi et al. (2008) Clin. Cancer Res. 14(9):2775). A recent trial utilizing IL-2 activated, killer immunoglobulin-like receptor-ligand mismatched natural killer (“NK”) cell transfusions from haplo-identical donors yielded a near complete response in 50% of multiple myeloma patients (Shi et al. (2008) Brit. J. Haemotol. 143:641). However, 5 of the 10 patients relapsed early (31-133 days) after NK cell infusion and 2 had progressive disease, which could have been due to an insufficient dose of NK cells or early rejection. Furthermore, appropriate NK cell donors were found for only 30% of patients who were otherwise eligible for the trial.

Accordingly, described herein are methods of treating multiple myeloma by administering to a patient in need thereof a therapeutically effective amount of an antibody targeted to myeloma cells or an antigen binding fragment thereof, or an antibody-drug conjugate, and a therapeutically effective amount of expanded and activated autologous NK cells. Also described herein are methods of treating multiple myeloma by administering to a patient in need thereof a therapeutically effective amount of an antibody targeted to the killer-cell immunoglobulin-like receptor (“KIR,” the NK inhibitory receptor) on NK cells or an antigen binding fragment thereof, and a therapeutically effective amount of expanded and activated autologous NK cells. The therapies described herein can be administered with other therapeutic agents, for example in combination with chemotherapeutic agents. Specific therapeutic regimens are provided herein. Patients with multiple myeloma at any stage can benefit from treatments in accordance with the methods described herein.

All publications mentioned in this specification are herein incorporated by reference. Any discussion of documents, acts, materials, devices, articles or the like that has been included in this specification is solely for the purpose of providing a context for the present disclosure. It is not to be taken as an admission that any or all of these matters form part of the prior art base or were common general knowledge in the field relevant to the present disclosure as it existed anywhere before the priority date of this application.

The features and advantages of the disclosure will become further apparent from the following detailed description of embodiments thereof.

It should be noted that the indefinite articles “a” and “an” and the definite article “the” are used in the present application, as is common in patent applications, to mean one or more unless the context clearly dictates otherwise. Further, the term “or” is used in the present application, as is common in patent applications, to mean the disjunctive “or” or the conjunctive “and.”

### 3. BRIEF DESCRIPTION OF THE FIGURES

FIG. 1 shows the percentage of NK cells, T-cells and NKT cells present at 0, 7 and 14 days of ex vivo co-culture of PBMCs from myeloma patients with K562-mb15-41BBL cells.

FIG. 2 shows the fold-increase in the number of NK cells and T-cells from four patients with multiple myeloma after 14-days of co-culturing with K562-mb15-41BBL cells.

FIG. 3 demonstrates the level of expression of CD3 and CD56 on the surface of NK cells from four patients with multiple myeloma before and after ex vivo expansion.

FIG. 4 shows the immunophenotype of expanded NK cells from multiple myeloma patients.

FIG. 5 shows in vitro specific lysis of cells from multiple myeloma patients upon exposure to non-expanded and expanded autologous NK cells. Multiple myeloma cells were treated as follows: (i) with non-expanded NK cells or expanded NK cells alone; (ii) with elotuzumab followed by non-expanded NK cells or expanded NK cells; or (iii) with an isotype control antibody followed by non-expanded NK cells or expanded NK cells.

FIG. 6 shows the distribution of expanded NK cells from multiple myeloma patients in the bodies of NOD-SKID mice at 0, 4 and 48 hours after injection into the tail vein.

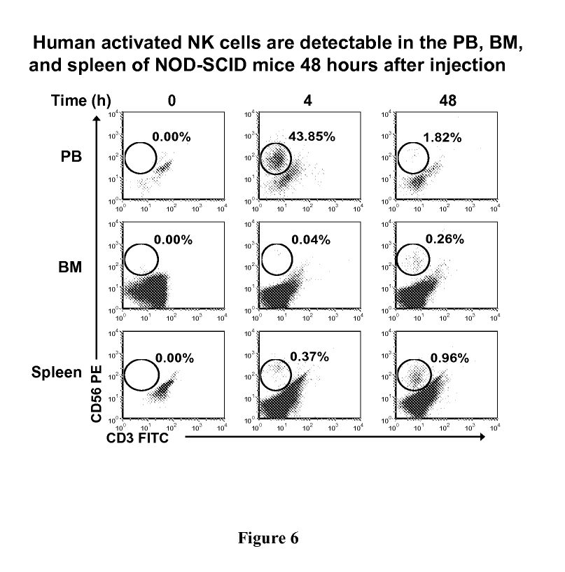

### 4. DETAILED DESCRIPTION

The present disclosure relates to compositions and methods for treating multiple myeloma in a subject. Specifically, the present disclosure relates to the treatment of multiple myeloma in a subject by administering an effective amount of autologous effector cells, in particular, autologous NK cells, and an effective amount of an antibody that targets multiple myeloma cells, or an antigen binding fragment thereof, or an antibody-drug conjugate, and elicits antibody-dependent cellular cytoxicity (ADCC). As used herein, the phrases “an antibody that targets multiple myeloma cells” or “myeloma cell targeting antibody” refers to an antibody that binds to an antigen present on the surface of myeloma cells. In some embodiments, the antigen is more highly expressed on myeloma cells than on non-cancerous cells. In various embodiments, the antibody that targets multiple myeloma cells is selected from an anti-CS1 antibody, an anti-CD20 antibody, an anti-CD38 antibody, an anti-CD40 antibody, an anti-CD56 antibody, an anti-CD74 antibody, an anti-CD138 antibody, an anti-CD317 antibody, an anti-IGF receptor antibody, an anti-IL-6 receptor antibody, or an anti-TRAIL receptor (including TRAIL receptor 1 and TRAIL receptor 2) antibody i.e., is an antibody that binds to CS1, CD20 (such as rituximab), CD38, CD40 (such as HCD122 or SGN-40), CD56 (such as huN901-DM1), CD74 (such as HLL1), CD138, CD317 (also known as HM1.24 antigen), IGF receptor (such as CP-751,871), IL-6 receptor (such as atlizumab, tocilizumab), or TRAIL receptor (such as mapatumumab or lexatumumab) expressed on the myeloma cell surface. In particular, the present disclosure relates to the treatment of multiple myeloma in a subject with a combination of expanded autologous NK cells and an anti-CS1 antibody. In various embodiments, the anti-CS1 antibody is elotuzumab (HuLuc63).

In various embodiments, the present disclosure relates to the treatment of multiple myeloma in a subject by administering an effective amount of autologous effector cells, in particular, autologous NK cells, and an effective amount of an antibody that targets NK cells and elicits increased ADCC toward myeloma cells. In particular, the present disclosure relates to the treatment of multiple myeloma in a subject by administering an effective amount of autologous NK cells and an effective amount of an antibody that targets the KIR protein on the surface of NK cells. In some embodiments, the present disclosure relates to the treatment of multiple myeloma in a subject by administering an effective amount of autologous NK cells, an effective amount of an antibody that targets myeloma cells, and an effective amount of an antibody that targets the KIR protein on NK cells.

A “subject” or “patient” to whom the combination therapy is administered can be a mammal, such as a non-primate (e.g., cow, pig, horse, cat, dog, rat, etc.) or a primate (e.g., monkey or human).

Treatment of multiple myeloma includes the treatment of patients already diagnosed as having any form of the disease at any clinical stage or manifestation; the delay of the onset or evolution or aggravation or deterioration of the symptoms or signs of the disease; and/or preventing and/or reducing the severity of the disease.

4.1 Autologous Effector Cells

The present disclosure relates to the use of expanded autologous effector cells from a subject with multiple myeloma in combination with an antibody that targets myeloma cells and/or an antibody that targets the KIR protein on NK cells and elicits ADCC to treat multiple myeloma. In certain embodiments, the effector cells for use in the methods of the disclosure are autologous lymphoid cells, i.e., lymphoid cells from the subject to be treated. In particular embodiments, the autologous lymphoid cells are natural killer (“NK”) cells.

In certain embodiments, NK cells are obtained from peripheral blood mononuclear cells (“PMBCs”) of the subject to be treated. In particular embodiments, the NK cells are expanded. The term “expanded” as used herein in the context of effector cells (i.e., NK cells) refers to effector cells that are cultured under conditions that promote (i) an increase in the total number of effector cells relative to the number in the starting culture and (ii) the activation of the effector cells. The terms “activate” or “activated” as used herein in relation to effector cells refer to inducing a change in their biologic state by which the cells express activation markers, produce cytokines, proliferate and/or become cytotoxic to target cells. Typically, NK cells are expanded and activated under the culturing conditions described herein. In particular embodiments, culturing conditions used to expand and activate NK cells in a mixed culture (e.g., PMBCs) promote activation of NK cells but not of T-cells or NKT-cells.

In certain embodiments, PBMCs are cultured under conditions that promote an increase in the fraction of NK cells and a decrease in the fraction of T-cells and/or NKT cells relative to the starting culture. In some embodiments, PBMCs are cultured under conditions that promote an increase in the fraction of NK cells in the culture and no increase or decrease in the fraction of T-cells and/or NKT cells in the culture relative to the starting culture. In particular embodiments, PBMCs are cultured under conditions that promote expansion of NK cells so that NK cells are the largest fraction of cells in the culture. In various embodiments, NK cells lacking T-cell receptors (CD56⁺ CD3⁻ cells) are preferentially expanded.

In some embodiments, NK cells are at least about 10% of the total cell population at the end of the culturing period. In various embodiments, NK cells are at least about 15% of the total cell population, such as at least about 20%, such as at least about 25%, such as at least about 30%, such as at least about 35%, such as at least about 40%, such as at least about 45%, such as at least about 50%, such as at least about 55%, such as at least about 60%, such as at least about 65%, such as at least about 70%, such as at least about 75%, such as at least about 80%, such as at least about 85%, such as at least about 90%, such as at least about 95%, such as at least about 96%, such as at least about 97%, such as at least about 98%, or such as at least about 99% of the total cell population at the end of the culturing period, or a percentage of the total cell population ranging between any of the foregoing values (e.g., NK cells are from at least about 50% to at least about 70% of the total cell population at the end of the culturing period).

In particular embodiments, NK cell expansion is about 10-fold at the end of the culturing period relative to the number of NK cells in the starting cell culture. In various embodiments, NK cell expansion is at least about 15-fold, such as at least about 20-fold, such as at least about 25-fold, such as at least about 30-fold, such as at least about 35-fold, such as at least about 40-fold, such as at least about 45-fold, such as at least about 50-fold, such as at least about 55-fold, such as at least about 60-fold, such as at least about 65-fold, such as at least about 70-fold, such as at least about 75-fold, such as at least about 80-fold, such as at least about 85-fold, such as at least about 90-fold, such as at least about 95-fold, such as at least about 100-fold, such as at least about 150-fold, such as at least about 200-fold, such as at least about 250-fold, such as at least about 300-fold, such as at least about 350-fold, such as at least about 400-fold, such as at least about 500-fold, such as at least about 600-fold, such as at least about 750-fold, such as at least about 1000-fold, such as at least about 5000-fold, such as at least about 7500-fold, such as at least about 10,000-fold or more at the end of the culturing period relative to the number of NK cells in the starting culture, or a fold-value ranging between any of the foregoing values (e.g., NK cell expansion is from at least about 95-fold to at least about 200-fold at the end of the culturing period).

Expansion and activation of NK cells can be accomplished by any method known in the art. (See e.g, Cho et al. (2009) Korean J. Lab. Med. 29:89 and U.S. Patent Publication No. 2006/0093605, each of which is incorporated herein by reference in its entirety). In some embodiments, NK cells, e.g., in PBMCs, are cultured in the presence of stimulatory cytokines. Such cytokines include, but are not limited to, IL-2, IL-4, IL-7, IL-12 and IL-15, either alone or in combination. In other embodiments, NK cells are expanded and activated by culturing the cells in the presence of stimulatory molecules such as an anti-CD3 antibody and IL-2.

Expansion and activation of NK cells can also be accomplished by co-culturing the cells with accessory cells. In certain embodiments, such accessory cells include, but are not limited to, monocytes, B-lymphblastoid cells, HFWT cells (a Wilms tumor-derived cell line), allogeneic mononuclear cells, autologous lymphocytes, mitogen activated lymphocytes and umbilical cord mesenchymal cells. In various embodiments, the accessory cells are K562 cells, a cell line derived from a patient with myeloid blast crisis of chronic myelogenous leukemia and bearing the BCR-ABL1 translocation. In certain embodiments, NK cells are co-cultured with accessory cells alone or in the presence of one or more cytokines. In certain embodiments, the cytokines are added to the culture medium. In other embodiments, the cytokines are expressed on the surface of the accessory cells.

In some embodiments, expansion and activation of NK cells are accomplished by co-culturing with accessory cells that have been modified to express NK stimulatory molecules on the cell surface. In certain embodiments, the stimulatory molecules include 4-1BBL (the ligand for 4-1BB, which is also known as CD137L), and membrane bound IL-15. In some embodiments, cell lines that can be modified for use as accessory cells to expand and activate NK cells include, but are not limited to, K562 cells, HFWT cells, HHUA cells (uterine endometrium cell line), HMV-II (melanoma cell line), HuH-6 (hepatoblastoma cell line), Lu-130 and Lu-134-A (small cell lung carcinoma cell lines), NB19 and NB69 (neuroblastoma cell lines), NEC14 (embryonal carcinoma cell line), TCO-2 (cervical carcinoma cell line) and TNB1 (neuroblastoma cell line). In particular embodiments, the cell line used as accessory cells in co-culture does not express or poorly expresses both MHC I and MHC II molecules. In certain embodiments, the accessory cells are K562 cells modified to express 4-1BBL and membrane-bound IL-15. In some embodiments, the accessory cell is K562-mb15-41BBL. (See Cho et al. (2009) Korean J. Lab. Med. 29:89-96, which is incorporated herein by reference in its entirety).

In some embodiments, the co-culture is started with a 1:1 ratio of accessory cells to CD56⁺CD3⁻ cells in the culture. In other embodiments, the co-culture is started with a 2:1 ratio, a 3:1 ratio, a 4:1 ratio, a 5:1 ratio, a 6:1 ratio, a 7:1 ratio, an 8:1 ratio, a 9:1 ratio, a 10:1 ratio, an 11:1 ratio, a 12:1 ratio, a 13:1 ratio, a 14:1 ratio or a 15:1 ratio of accessory cells to CD56⁺CD3⁻ cells in the culture. The number of viable CD56⁺CD3⁻ cells in a culture can be quantified by any method known in the art, including, but not limited to, Trypan-blue dye exclusion and by flow cytometry using labeled antibodies for CD56. In certain embodiments, co-cultures are maintained for less than 24 hours, such as for about 4 hours, about 6 hours, about 8 hours, about 10 hours, about 12 hours, about 14 hours, about 16 hours, about 18 hours or about 20 hours. In other embodiments, co-cultures are maintained for about 1 week, for about 2 weeks or for about 3 weeks. In some embodiments, co-cultures are maintained for a period of time ranging between any two of the foregoing values (e.g., co-cultures are maintained for about 8 hours to about 18 hours). In particular embodiments, co-cultures are maintained for 2 weeks. It will be understood by the skilled artisan that prolonging the time of co-culture will increase the number of autologous NK cells. Thus, it is within the skill in the art to adjust the time of co-culture based on the desired level of expansion and activation of the NK cells. In various embodiments, in order to prevent overgrowth of accessory cells, the co-culture is irradiated at doses of, e.g., 30 Gy, 50 Gy, 70 Gy, or 100 Gy.

NK cells can be expanded using reagents and culture conditions known in the art. An exemplary protocol for obtaining clinical-grade purified functional NK cells for infusion is set forth in Cho et al. (2009) Korean J. Lab. Med. 29:89-96 and the references cited therein, which is incorporated herein by reference in its entirety.

In certain embodiments, activated NK cells are genetically modified after expansion to express artificial receptors directed against molecules that are present on the surface of cancer cells. In various embodiments, NK cells are re-stimulated after genetic modification, e.g., by co-culturing the genetically modified NK cells with accessory cells. Such genetic modification of activated NK cells can be accomplished by any method known in the art. In some embodiments, genetic modification of NK cells can be accomplished by transduction with retroviruses carrying plasmids that encode artificial receptor molecules. (See, e.g., U.S. Patent Publication No. 2006/0093605 and Imai et al. (2005) Blood 106:376-383, each of which is incorporated herein by reference in its entirety).

In some embodiments, a solid support may be used to expand and activate NK cells instead of accessory cells expressing stimulatory molecules on the cell surface. In certain embodiments, such supports will have attached on the surface one or more molecules capable of binding to NK cells and inducing activation or a proliferative response. In some embodiments, the supports are designed to bind one or more molecules that induce activation of NK cells or a proliferative response when NK cells are passed over the solid support and bind to the one or more molecules. Molecules that induce activation of or a proliferative response from NK cells include, but are not limited to CD137, IL-15, or fragments of either CD137 or IL-15 that retain the ability to induce the desired response. See U.S. Patent Publication No. 2006/0093605, which is incorporated herein by reference in its entirety.

4.2 Antibodies Targeting Multiple Myeloma Cells or NK Cells

Unless indicated otherwise, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bispecific antibodies, diabodies, triabodies, and tetrabodies), and antigen binding fragments of antibodies, including e.g., Fab′, F(ab′)2, Fab, Fv, rIgG, and scFv fragments. Moreover, unless otherwise indicated, the term “monoclonal antibody” (mAb) is meant to include both intact molecules, as well as, antibody fragments (such as, for example, Fab and F(ab′)2 fragments) which are capable of specifically binding to a protein. Fab and F(ab′)2 fragments lack the Fc fragment of intact antibody, clear more rapidly from the circulation of the animal or plant, and may have less non-specific tissue binding than an intact antibody (Wahl et al. (1983) J. Nucl. Med. 24:316).

In some embodiments, the antibodies, or an antigen binding fragment thereof, or an antibody-drug conjugate, for use in the methods described herein are directed to a protein that is expressed on the surface of multiple myeloma cells. In certain embodiments, the antibodies are directed to a protein selected from CS1, CD20, CD38, CD40, CD56, CD74, CD138, CD317, IGF receptor, IL-6 receptor and TRAIL receptor. In various embodiments, the antibody is the anti-CD20 antibody rituximab. In some embodiments, the anti-CD40 antibody is selected from HCD122 and SGN-40. In certain embodiments the anti-CD56 antibody is huN901-DM1. In some embodiments the anti-CD74 antibody is HLL1. In still other embodiments, the anti-IGF receptor antibody is CP-751,871. In some embodiments, the anti-IL-6 receptor antibody is selected from atlizumab and tocilizumab. In certain embodiments, the anti-TRAIL receptor antibody is selected from mapatumumab and lexatumumab. In other embodiments, the antibodies, or an antigen binding fragment thereof, for use in the methods described herein are directed to a protein that is expressed on the surface of NK cells, such as KIR.

In certain embodiments, the antibodies for use in the methods described herein are anti-CS1 antibodies. Anti-CS1 antibodies that are suitable for use in the methods of treatment disclosed herein include, but are not limited to, isolated antibodies that bind one or more of the three epitope clusters identified on CS1 and monoclonal antibodies produced by the hybridoma cell lines: Luc2, Luc3, Luc15, Luc22, Luc23, Luc29, Luc32, Luc34, Luc35, Luc37, Luc38, Luc39, Luc56, Luc60, Luc63, Luc69, LucX.1, LucX.2 or Luc90. These monoclonal antibodies are named as the antibodies: Luc2, Luc3, Luc15, Luc22, Luc23, Luc29, Luc32, Luc34, Luc35, Luc37, Luc38, Luc39, Luc56, Luc60, Luc63, Luc69, LucX and Luc90, respectively, hereafter. Humanized versions are denoted by the prefix “hu” or “Hu” (see, e.g., U.S. Patent Publication Nos. 2005/0025763 and 2006/0024296, the contents of which are incorporated herein by reference).

In certain embodiments, suitable anti-CS1 antibodies include antibodies that bind one or more of the three epitope clusters identified on CS1 (SEQ ID NO: 1, Table 1 below; see, e.g., U.S. Patent Publication No. 2006/0024296, the content of which is incorporated herein by reference). As disclosed in U.S. Patent Publication No. 2006/0024296, the CS1 antibody binding sites have been grouped into 3 epitope clusters:
the epitope cluster defined by Luc90, which binds to hu50/mu50. This epitope covers from about amino acid residue 23 to about amino acid residue 151 of human CS1. This epitope is resided within the domain 1 (V domain) of the extracellular domain. This epitope is also recognized by Luc34, LucX (including LucX1 and LucX2) and Luc69;
the epitope cluster defined by Luc38, which binds to mu25/hu75 and hu50/mu50. This epitope likely covers from about amino acid residue 68 to about amino acid residue 151 of human CS1. This epitope is also recognized by Luc5; and
the epitope cluster defined by Luc63, which binds to mu75/hu25. This epitope covers from about amino acid residue 170 to about amino acid residue 227 of human CS1. This epitope is resided within domain 2 (C2 domain) of human CS1. This epitope is also recognized by Luc4, Luc 12, Luc23, Luc29, Luc32 and Luc37.

In a specific example, the anti-CS1 antibody used in the present methods is Luc63 or comprises the light chain variable region and/or heavy chain variable region sequence of Luc63. The amino acid sequences for the heavy chain variable region and the light chain variable region for Luc63 are disclosed in U.S. Patent Publication No. 2005/0025763 as SEQ ID NO:5 and SEQ ID NO:6, respectively, the contents of which are incorporated herein by reference. The sequences of the heavy and light chain variable regions of Luc63 are represented herein by SEQ ID NO:1 and SEQ ID NO:2, respectively. In other aspects, the anti-CS1 antibody used in the treatment of multiple myeloma comprises the heavy chain CDR sequences, light chain CDR sequences, or both heavy and light chain CDR sequences of Luc63, or comprises one, two or three CDR sequences having at least 80%, at least 85%, or at least 90% sequence identity to the heavy chain CDR sequences, light chain CDR sequences, or both heavy and light chain CDR sequences of Luc63. The heavy chain CDR sequences of Luc63 are represented herein by SEQ ID NOS. 3, 4 and 5, and the light chain CDR sequence of Luc63 are represented herein by SEQ ID NOS. 6, 7 and 8.

In a specific example, the anti-CS1 antibody used in the present methods is HuLuc63 or comprises the light chain variable region and/or heavy chain variable region sequence of HuLuc63. The amino acid sequences for the heavy chain variable region and the light chain variable region for HuLuc63 are disclosed in U.S. Patent Publication No. 2006/0024296 as SEQ ID NO:41 and SEQ ID NO:44, respectively, the contents of which are incorporated herein by reference. The sequences of the heavy and light chain variable regions of HuLuc63 are represented herein by SEQ ID NO:9 and SEQ ID NO:10, respectively. In other aspects, the anti-CS1 antibody used in the treatment of multiple myeloma comprises the heavy chain CDR sequences, light chain CDR sequences, or both heavy and light chain CDR sequences of HuLuc63, or comprises one, two or three CDR sequences having at least 80%, at least 85%, or at least 90% sequence identity to the heavy chain CDR sequences, light chain CDR sequences, or both heavy and light chain CDR sequences of HuLuc63. The heavy chain CDR sequences of HuLuc63 are represented herein by SEQ ID NOS. 11, 12 and 13, and the light chain CDR sequences of HuLuc63 are represented herein by SEQ ID NOS. 14, 15 and 16.

In another specific example, the anti-CS1 antibody used in the present methods is Luc90 or comprises the light chain variable region and/or heavy chain variable region sequence of Luc90. The amino acid sequences for the heavy chain variable region and the light chain variable region for Luc90 are disclosed in U.S. Patent Publication No. 2005/0025763 as SEQ ID NO:3 and SEQ ID NO:4, respectively, the contents of which are incorporated herein by reference. The sequences of the heavy and light chain variable regions of Luc90 are represented herein by SEQ ID NO:17 and SEQ ID NO:18, respectively. In other aspects, the anti-CS1 antibody used in the treatment of multiple myeloma comprises the heavy chain CDR sequences, light chain CDR sequences, or both heavy and light chain CDR sequences of Luc90, or comprises one, two or three CDR sequences having at least 80%, at least 85%, or at least 90% sequence identity to the heavy chain CDR sequences, light chain CDR sequences, or both heavy and light chain CDR sequences of Luc90. The heavy chain CDR sequences of Luc90 are represented herein by SEQ ID NOS. 19, 20 and 21, and the light chain CDR sequences of Luc90 are represented herein by SEQ ID NOS: 22, 23 and 24.

In yet another specific example, the anti-CS1 antibody used in the present methods is Luc34 or comprises the light chain variable region and/or heavy chain variable region sequence of Luc34. The amino acid sequences for the heavy chain variable region and the light chain variable region for Luc34 are disclosed in U.S. Patent Publication No. 2005/0025763 as SEQ ID NO:7 and SEQ ID NO:8, respectively, the contents of which are incorporated herein by reference. The sequences of the heavy and light chain variable regions of Luc34 are represented herein by SEQ ID NO:25 and SEQ ID NO:26, respectively. In other aspects, the anti-CS1 antibody used in the treatment of multiple myeloma comprises the heavy chain CDR sequences, light chain CDR sequences, or both heavy and light chain CDR sequences of Luc34, or comprises one, two or three CDR sequences having at least 80%, at least 85%, or at least 90% sequence identity to the heavy chain CDR sequences, light chain CDR sequences, or both heavy and light chain CDR sequences of Luc34. The heavy chain CDR sequences of Luc34 are represented herein by SEQ ID NOS. 27, 28 and 29, and the light chain CDR sequences of Luc34 are represented herein by SEQ ID NOS. 30, 31 and 32.

In yet another specific example, the anti-CS1 antibody used in the present methods is the LucX antibody LucX.2 or comprises the light chain variable region and/or heavy chain variable region sequence of LucX.2. The amino acid sequences for the heavy chain variable region and the light chain variable region for LucX.2 are disclosed in U.S. Patent Publication No. 2006/0024296 as SEQ ID NO:66 and SEQ ID NO:67, respectively, the contents of which are incorporated herein by reference. The sequences of the heavy and light chain variable regions of LucX.2 are represented herein by SEQ ID NO:33 and SEQ ID NO:34, respectively. In other aspects, the anti-CS1 antibody used in the treatment of multiple myeloma comprises the heavy chain CDR sequences, light chain CDR sequences, or both heavy and light chain CDR sequences of LucX.2, or comprises one, two or three CDR sequences having at least 80%, at least 85%, or at least 90% sequence identity to the heavy chain CDR sequences, light chain CDR sequences, or both heavy and light chain CDR sequences of LucX.2. The heavy chain CDR sequences of LucX.2 are represented herein by SEQ ID NOS. 35, 36 and 37, and the light chain CDR sequences of LucX.2 are represented herein by SEQ ID NOS. 38, 39 and 40.

Table 1 below provides the sequences of HuLuc63, Luc90, Luc34 and LucX.2 identified above:

TABLE 1
Light and heavy chain variable region sequences (in three-letter code) and CDR sequences (in single
letter code) of anti-CS1 antibodies useful for treatment of rare lymphomas.

| SEQ ID | | |
|---|---|---|
| NO. | Description | Sequence |
| 1 | Luc63 heavy chain variable | Glu Val Lys Leu Leu Glu Ser Gly Gly Gly Leu Val Gln Pro Gly Gly |
| | region | 1 5 10 15 |
| | | Ser Leu Lys Leu Ser Cys Ala Ala Ser Gly Phe Asp Phe Ser Arg Tyr |
| | | 20 25 30 |
| | | Trp Met Ser Trp Val Arg Gln Ala Pro Gly Lys Gly Leu Glu Trp Ile |
| | | 35 40 45 |
| | | Gly Glu Ile Asn Pro Asp Ser Ser Thr Ile Asn Tyr Thr Pro Ser Leu |
| | | 50 55 60 |
| | | Lys Asp Lys Phe Ile Ile Ser Arg Asp Asn Ala Lys Asn Thr Leu Tyr |
| | | 65 70 75 80 |
| | | Leu Gln Met Ser Lys Val Arg Ser Glu Asp Thr Ala Leu Tyr Tyr Cys |
| | | 85 90 95 |
| | | Ala Arg Pro Asp Gly Asn Tyr Trp Tyr Phe Asp Val Trp Gly Ala Gly |
| | | 100 105 110 |
| | | Thr Thr Val Thr Val Ser Ser |
| | | 115 |
| 2 | Luc63 light chain variable | Asp Ile Val Met Thr Gln Ser His Lys Phe Met Ser Thr Ser Val Gly |
| | region | 1 5 10 15 |
| | | Asp Arg Val Ser Ile Thr Cys Lys Ala Ser Gln Asp Val Gly Ile Ala |
| | | 20 25 30 |
| | | Val Ala Trp Tyr Gln Gln Lys Pro Gly Gln Ser Pro Lys Leu Leu Ile |
| | | 35 40 45 |
| | | Tyr Trp Ala Ser Thr Arg His Thr Gly Val Pro Asp Arg Phe Thr Gly |
| | | 50 55 60 |
| | | Ser Gly Ser Gly Thr Asp Phe Thr Leu Thr Ile Ser Asn Val Gln Ser |
| | | 65 70 75 80 |
| | | Glu Asp Leu Ala Asp Tyr Phe Cys Gln Gln Tyr Ser Ser Tyr Pro Tyr |
| | | 85 90 95 |
| | | Thr Phe Gly Gly Gly Thr Lys Leu Glu Ile Lys |
| | | 100 105 |
| 3 | Luc63 heavy chain variable | RYWMS |
| | region CDR1 | |
| 4 | Luc63 heavy chain variable | EINPDSSTINYTPSLKD |
| | region CDR2 | |
| 5 | Luc63 heavy chain variable | PDGNYWYFDV |
| | region CDR3 | |
| 6 | Luc63 light chain variable | KASQDVGIAVA |
| | region CDR1 | |
| 7 | Luc63 light chain variable | WASTRHT |
| | region CDR2 | |
| 8 | Luc63 light chain variable | QQYSSYPYT |
| | region CDR3 | |
| 9 | HuLuc63 heavy chain | Glu Val Gln Leu Val Glu Ser Gly Gly Gly Leu Val Gln Pro Gly Gly |
| | variable region | 1 5 10 15 |
| | | Ser Leu Arg Leu Ser Cys Ala Ala Ser Gly Phe Asp Phe Ser Arg Tyr |
| | | 20 25 30 |
| | | Trp Met Ser Trp Val Arg Gln Ala Pro Gly Lys Gly Leu Glu Trp Ile |
| | | 35 40 45 |
| | | Gly Glu Ile Asn Pro Asp Ser Ser Thr Ile Asn Tyr Ala Pro Ser Leu |
| | | 50 55 60 |
| | | Lys Asp Lys Phe Ile Ile Ser Arg Asp Asn Ala Lys Asn Ser Leu Tyr |
| | | 65 70 75 80 |
| | | Leu Gln Met Asn Ser Leu Arg Ala Glu Asp Thr Ala Val Tyr Tyr Cys |
| | | 85 90 95 |
| | | Ala Arg Pro Asp Gly Asn Tyr Trp Tyr Phe Asp Val Trp Gly Gln Gly |
| | | 100 105 110 |
| | | Thr Leu Val Thr Val Ser Ser |
| | | 115 |
| 10 | HuLuc63 light chain | Asp Ile Gln Met Thr Gln Ser Pro Ser Ser Leu Ser Ala Ser Val Gly |
| | variable region | 1 5 10 15 |
| | | Asp Arg Val Thr Ile Thr Cys Lys Ala Ser Gln Asp Val Gly Ile Ala |
| | | 20 25 30 |
| | | Val Ala Trp Tyr Gln Gln Lys Pro Gly Lys Val Pro Lys Leu Leu Ile |
| | | 35 40 45 |
| | | Tyr Trp Ala Ser Thr Arg His Thr Gly Val Pro Asp Arg Phe Ser Gly |
| | | 50 55 60 |
| | | Ser Gly Ser Gly Thr Asp Phe Thr Leu Thr Ile Ser Ser Leu Gln Pro |
| | | 65 70 75 80 |
| | | Glu Asp Val Ala Thr Tyr Tyr Cys Gln Gln Tyr Ser Ser Tyr Pro Tyr |
| | | 85 90 95 |
| | | Thr Phe Gly Gln Gly Thr Lys Val Glu Ile Lys |
| | | 100 105 |
| 11 | HuLuc63 heavy chain | RYWMS |
| | variable region CDR1 | |
| 12 | HuLuc63 heavy chain | EINPDSSTINYAPSLKD |
| | variable region CDR2 | |
| 13 | HuLuc63 heavy chain | PDGNYWYFDV |
| | variable region CDR3 | |
| 14 | HuLuc63 light chain | KASQDVGIAVA |
| | variable region CDR1 | |
| 15 | HuLuc63 light chain | WASTRHT |
| | variable region CDR2 | |
| 16 | HuLuc63 light chain | QQYSSYPYT |
| | variable region CDR3 | |
| 17 | Luc90 heavy chain variable | Gln Val Gln Leu Gln Gln Pro Gly Ala Glu Leu Val Arg Pro Gly Ala |
| | region | 1 5 10 15 |
| | | Ser Val Lys Leu Ser Cys Lys Ala Ser Gly Tyr Ser Phe Thr Thr Tyr |
| | | 20 25 30 |
| | | Trp Met Asn Trp Val Lys Gln Arg Pro Gly Gln Gly Leu Glu Trp Ile |
| | | 35 40 45 |
| | | Gly Met Ile His Pro Ser Asp Ser Glu Thr Arg Leu Asn Gln Lys Phe |
| | | 50 55 60 |
| | | Lys Asp Lys Ala Thr Leu Thr Val Asp Lys Ser Ser Ser Thr Ala Tyr |
| | | 65 70 75 80 |
| | | Met Gln Leu Ser Ser Pro Thr Ser Glu Asp Ser Ala Val Tyr Tyr Cys |
| | | 85 90 95 |
| | | Ala Arg Ser Thr Met Ile Ala Thr Arg Ala Met Asp Tyr Trp Gly Gln |
| | | 100 105 110 |
| | | Gly Thr Ser Val Thr Val Ser Ser |
| | | 115 120 |
| 18 | Luc90 light chain variable | Asp Ile Val Met Thr Gln Ser Gln Lys Ser Met Ser Thr Ser Val Gly |
| | region | 1 5 10 15 |
| | | Asp Arg Val Ser Ile Thr Cys Lys Ala Ser Gln Asp Val Ile Thr Gly |
| | | 20 25 30 |
| | | Val Ala Trp Tyr Gln Gln Lys Pro Gly Gln Ser Pro Lys Leu Leu Ile |
| | | 35 40 45 |
| | | Tyr Ser Ala Ser Tyr Arg Tyr Thr Gly Val Pro Asp Arg Phe Thr Gly |
| | | 50 55 60 |
| | | Ser Gly Ser Gly Thr Asp Phe Thr Phe Thr Ile Ser Asn Val Gln Ala |
| | | 65 70 75 80 |
| | | Glu Asp Leu Ala Val Tyr Tyr Cys Gln Gln His Tyr Ser Thr Pro Leu |
| | | 85 90 95 |
| | | Thr Phe Gly Ala Gly Thr Lys Leu Glu Leu Lys |
| | | 100 105 |
| 19 | Luc90 heavy chain variable | TYWMN |
| | region CDR1 | |
| 20 | Luc90 heavy chain variable | MIHPSDSETRLNQKFKD |
| | region CDR2 | |
| 21 | Luc90 heavy chain variable | STMIATRAMDY |
| | region CDR3 | |
| 22 | Luc90 light chain variable | KASQDVITGVA |
| | region CDR1 | |
| 23 | Luc90 light chain variable | SASYRYT |
| | region CDR2 | |
| 24 | Luc90 light chain variable | QQHYSTPLT |
| | region CDR3 | |
| 25 | Luc34 heavy chain variable | Gln Val Gln Leu Gln Gln Ser Gly Ala Glu Leu Ala Arg Pro Gly Ala |
| | region | 1 5 10 15 |
| | | Ser Val Lys Leu Ser Cys Lys Ala Ser Gly Tyr Thr Phe Thr Ser Tyr |
| | | 20 25 30 |
| | | Trp Met Gln Trp Val Lys Gln Arg Pro Gly Gln Gly Leu Glu Trp Ile |
| | | 35 40 45 |
| | | Gly Ala Ile Tyr Pro Gly Asp Gly Asp Thr Arg Tyr Thr Gln Lys Phe |
| | | 50 55 60 |
| | | Lys Gly Lys Ala Thr Leu Thr Ala Asp Lys Ser Ser Ser Thr Ala Tyr |
| | | 65 70 75 80 |
| | | Met Gln Leu Ser Ser Leu Ala Ser Glu Asp Ser Ala Val Tyr Tyr Cys |
| | | 85 90 95 |
| | | Ala Arg Gly Lys Val Tyr Tyr Gly Ser Asn Pro Phe Ala Tyr Trp Gly |
| | | 100 105 110 |
| | | Gln Gly Thr Leu Val Thr Val Ser Ala |
| | | 115 120 |
| 26 | Luc34 light chain variable | Asp Ile Gln Met Thr Gln Ser Ser Ser Tyr Leu Ser Val Ser Leu Gly |
| | region | 1 5 10 15 |
| | | Gly Arg Val Thr Ile Thr Cys Lys Ala Ser Asp His Ile Asn Asn Trp |
| | | 20 25 30 |
| | | Leu Ala Trp Tyr Gln Gln Lys Pro Gly Asn Ala Pro Arg Leu Leu Ile |
| | | 35 40 45 |
| | | Ser Gly Ala Thr Ser Leu Glu Thr Gly Val Pro Ser Arg Phe Ser Gly |
| | | 50 55 60 |
| | | Ser Gly Ser Gly Lys Asp Tyr Thr Leu Ser Ile Thr Ser Leu Gln Thr |
| | | 65 70 75 80 |
| | | Glu Asp Val Ala Thr Tyr Tyr Cys Gln Gln Tyr Trp Ser Thr Pro Trp |
| | | 85 90 95 |
| | | Thr Phe Gly Gly Gly Thr Lys Leu Glu Ile Lys |
| | | 100 105 |
| 27 | Luc34 heavy chain variable | SYWMQ |
| | region CDR1 | |
| 28 | Luc34 heavy chain variable | AIYPGDGDTRYTQKFKG |
| | region CDR2 | |
| 29 | Luc34 heavy chain variable | GKVYYGSNPFAY |
| | region CDR3 | |
| 30 | Luc34 light chain variable | KASDHINNWLA |
| | region CDR1 | |
| 31 | Luc34 light chain variable | GATSLET |
| | region CDR2 | |
| 32 | Luc34 light chain variable | QQYWSTPWT |
| | region CDR3 | |
| 33 | LucX.2 heavy chain | Gln Val Gln Leu Gln Gln Ser Gly Pro Glu Leu Val Lys Pro Gly Ala |
| | variable region | 1 5 10 15 |
| | | Ser Val Lys Ile Ser Cys Lys Ala Ser Gly Tyr Ala Phe Ser Ser Ser |
| | | 20 25 30 |
| | | Trp Met Asn Trp Val Lys Gln Arg Pro Gly Gln Gly Leu Glu Trp Ile |
| | | 35 40 45 |
| | | Gly Arg Ile Tyr Pro Gly Asp Gly Asp Thr Lys Tyr Asn Gly Lys Phe |
| | | 50 55 60 |
| | | Lys Gly Lys Ala Thr Leu Thr Ala Asp Lys Ser Ser Ser Thr Ala Tyr |
| | | 65 70 75 80 |
| | | Met Gln Leu Ser Ser Leu Thr Ser Val Asp Ser Ala Val Tyr Phe Cys |
| | | 85 90 95 |
| | | Ala Arg Ser Thr Met Ile Ala Thr Gly Ala Met Asp Tyr Trp Gly Gln |
| | | 100 105 110 |
| | | Gly Thr Ser Val Thr Val Ser Ser |
| | | 115 120 |
| 34 | LucX.2 light chain variable | Asp Ile Val Met Thr Gln Ser His Lys Phe Met Ser Thr Ser Val Gly |
| | region | 1 5 10 15 |
| | | Asp Arg Val Ser Ile Thr Cys Lys Ala Ser Gln Asp Val Ser Thr Ala |
| | | 20 25 30 |
| | | Val Ala Trp Tyr Gln Gln Lys Pro Gly Gln Ser Pro Lys Leu Leu Ile |
| | | 35 40 45 |
| | | Tyr Ser Ala Ser Tyr Arg Tyr Thr Gly Val Pro Asp Arg Phe Thr Gly |
| | | 50 55 60 |
| | | Ser Gly Ser Gly Thr Asp Phe Thr Phe Thr Ile Ser Ser Val Gln Ala |
| | | 65 70 75 80 |
| | | Glu Asp Leu Ala Val Tyr Tyr Cys Gln Gln His Tyr Ser Thr Pro Pro |
| | | 85 90 95 |
| | | Tyr Thr Phe Gly Gly Gly Thr Lys Leu Glu Ile Lys |
| | | 100 105 |
| 35 | LucX.2 heavy chain | SSWMN |
| | variable region CDR1 | |
| 36 | LucX.2 heavy chain | RIYPGDGDTKYNGKFKG |
| | variable region CDR2 | |
| 37 | LucX.2 heavy chain | STMIATGAMDY |
| | variable region CDR3 | |
| 38 | LucX.2 light chain variable | KASQDVSTAVA |
| | region CDR1 | |
| 39 | LucX.2 light chain variable | SASYRYT |
| | region CDR2 | |
| 40 | LucX.2 light chain variable | QQHYSTPPYT |
| | region CDR3 | |

In certain embodiments, anti-CS1 antibodies useful in the methods disclosed herein compete with Luc63 or Luc90 for binding to CS1. The ability to compete for binding to CS1 can be tested using a competition assay. In one example of a competition assay, CS1 is adhered onto a solid surface, e.g., a microwell plate, by contacting the plate with a solution of CS1 (e.g., at a concentration of 5 μg/ml in PBS over night at 4° C.). The plate is washed and blocked (e.g., in TBS buffer with 5 mM CaCl₂ and 2% BSA). A solution of fluorescently labeled Luc63 or Luc90 (the “reference” antibody) (e.g., at a concentration of 1 μg/ml, 2 μg/ml, or 5 μg/ml) is added to the plate and plates are incubated for 2 hours. The plate is washed, the competing anti-CS1 antibody (the “test” antibody) is added (e.g., at a concentration of 3 μg/ml, 10 μg/ml, 20 μg/ml, 50 μg/ml or 100 μg/ml), and the plates incubated for 1 hour. The assay can be performed in parallel at different concentrations of competing antibody. Plates are washed and the mean fluorescence intensity (“MFI”) is measured as compared to control plates (which were not incubated with a test antibody, e.g., were incubated with an isotype control antibody). Variations on this neutralizing assay can also be used to test competition between Luc63 or Luc90 and another anti-CS1 antibody. For example, in certain aspects, the anti-CS1 antibody is used as a reference antibody and Luc63 or Luc90 is used as a test antibody. Additionally, instead of soluble CS1 membrane-bound CS1 can be used, for example recombinantly expressed on cells (preferably mammalian cells, e.g., COS cells) in culture. Generally, about 104 to 106 transfectants, and, in a specific embodiment, about 105 transfectants, are used. Other formats for competition assays are known in the art and can be employed. The hybridoma cell line producing the antibody Luc90 has been deposited with the American Type Culture Collection (ATCC) at P.O. Box 1549, Manassas, Va. 20108, as accession number PTA-5091. The deposit of this hybridoma cell line was received by the ATCC on Mar. 26, 2003. The hybridoma cell line Luc63 has also been deposited with the ATCC at the address listed above, as accession number PTA-5950. The deposit of the Luc63 antibody was received by the ATCC on May 6, 2004.

In various embodiments, an anti-CS1 antibody useful to treat multiple myeloma reduces the MFI of labeled Luc63 or Luc90 by at least 40%, by at least 50%, by at least 60%, by at least 70%, by at least 80%, by at least 90%, or by a percentage ranging between any two of the foregoing values (e.g., an anti-CS1 antibody reduces the MFI of labeled Luc63 or luc90 by 50% to 70%) when the anti-CS1 antibody is used at a concentration of 3 μg/ml, 10 μg/ml, 20 μg/ml, 50 μg/ml, 100 μg/ml, or at a concentration ranging between any two of the foregoing values (e.g., at a concentration ranging from 20 μg/ml to 50 μg/ml).

In other embodiments, Luc63 or Luc90 reduces the MFI of a labeled anti-CS1 antibody useful in the methods disclosed herein by at least 40%, by at least 50%, by at least 60%, by at least 70%, by at least 80%, by at least 90%, or by a percentage ranging between any two of the foregoing values (e.g., Luc63 or Luc90 reduces the MFI of a labeled an anti-CS1 antibody by 50% to 70%) when Luc63 or Luc90 is used at a concentration of 3 μg/ml, 10 μg/ml, 20 μg/ml, 50 μg/ml, 100 μg/ml, or at a concentration ranging between any two of the foregoing values (e.g., at a concentration ranging from 10 μg/ml to 50 μg/ml).

Anti-CS1 antibodies useful in the present methods include antibodies that induce antibody-dependent cytotoxicity (ADCC) of CS1-expressing cells. The ADCC of an anti-CS1 antibody can be improved by using antibodies that have low levels of or lack fucose. Antibodies lacking fucose have been correlated with enhanced ADCC (antibody-dependent cellular cytotoxicity) activity, especially at low doses of antibody (Shields et al. (2002) J. Biol. Chem. 277:26733; Shinkawa et al. (2003) J. Biol. Chem. 278:3466). Methods of preparing fucose-less antibodies include growth in rat myeloma YB2/0 cells (ATCC CRL 1662). YB2/0 cells express low levels of FUT8 mRNA, which encodes an enzyme (α1,6-fucosyltransferase) necessary for fucosylation of polypeptides. Alternative methods for increasing ADDC activity include mutations in the Fc portion of a CS1 antibody, particularly mutations which increase antibody affinity for an FcγR receptor. A correlation between increased FcγR binding with mutated Fc has been demonstrated using targeted cytoxicity cell-based assays (Shields et al. (2001) J. Biol. Chem. 276:6591; Presta et al. (2002) Biochem Soc. Trans. 30:487). Methods for increasing ADCC activity through specific Fc region mutations include the Fc variants comprising at least one amino acid substitution at a position selected from the group consisting of: 234, 235, 239, 240, 241, 243, 244, 245, 247, 262, 263, 264, 265, 266, 267, 269, 296, 297, 298, 299, 313, 325, 327, 328, 329, 330 and 332, wherein the numbering of the residues in the Fc region is that of the EU index as in Kabat (Kabat et al., Sequences of Proteins of Immunological Interest (National Institute of Health, Bethesda, Md. 1987)). In certain specific embodiments, said Fc variants comprise at least one substitution selected from the group consisting of L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L, F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, E269H, E269Y, E269F, E269R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, W313F, N325Q, N325L, N325I, N325D, N325E, N325A, N325T, N325V, N325H, A327N, A327L, L328M, L328D, L328E, L328N, L328Q, L328F, L328I, L328V, L328T, L328H, L328A, P329F, A330L, A330Y, A330V, A330I, A330F, A330R, A330H, I332D, I332E, I332N, I332Q, I332T, I332H, I332Y and I332A, wherein the numbering of the residues in the Fc region is that of the EU index as in Kabat. Fc variants can also be selected from the group consisting of V264L, V264I, F241W, F241L, F243W, F243L, F241L/F243L/V262I/V2641, F241W/F243W, F241W/F243W/V262A/V264A, F241L/V262I, F243L/V264I, F243L/V262I/V264W, F241Y/F243Y/V262T/V264T, F241E/F243R/V262E/V264R, F241E/F243Q/V262T/V264E, F241R/F243Q/V262T/V264R, F241E/F243Y/V262T/V264R, L328M, L328E, L328F, I332E, L3238M/I332E, P244H, P245A, P247V, W313F, P244H/P245A/P247V, P247G, V264I/I332E, F241E/F243R/V262E/V264R/I332E, F241E/F243Q/V262T/264E/I332E, F241R/F243Q/V262T/V264R/I332E, F241E/F243Y/V262T/V264R/I332E, S298A/I332E, S239E/I332E, S239Q/I332E, S239E, D265G, D265N, S239E/D265G, S239E/D265N, S239E/D265Q, Y296E, Y296Q, T299I, A327N, S267Q/A327S, S267L/A327S, A327L, P329F, A330L, A330Y, I332D, N297S, N297D, N297S/I332E, N297D/I332E, N297E/I332E, D265Y/N297D/I332E, D265Y/N297D/T299L/I332E, D265F/N297E/I332E, L328I/I332E, L328Q/I332E, I332N, I332Q, V264T, V264F, V240I, V263I, V266I, T299A, T299S, T299V, N325Q, N325L, N325I, S239D, S239N, S239F, S239D/I332D, S239D/I332E, S239D/I332N, S239D/I332Q, S239E/I332D, S239E/I332N, S239E/I332Q, S239N/I332D, S239N/I332E, S239N/I332N, S239N/I332Q, S239Q/I332D, S239Q/I332N, S239Q/I332Q, Y296D, Y296N, F241Y/F243Y/V262T/V264T/N297D/I332E, A330Y/I332E, V264I/A330Y/I332E, A330L/I332E, V264I/A330L/I332E, L234D, L234E, L234N, L234Q, L234T, L234H, L234Y, L234I, L234V, L234F, L235D, L235S, L235N, L235Q, L235T, L235H, L235Y, L235I, L235V, L235F, S239T, S239H, S239Y, V240A, V240T, V240M, V263A, V263T, V263M, V264M, V264Y, V266A, V266T, V266M, E269H, E269Y, E269F, E269R, Y296S, Y296T, Y296L, Y296I, A298H, T299H, A330V, A330I, A330F, A330R, A330H, N325D, N325E, N325A, N325T, N325V, N325H, L328D/I332E, L328E/I332E, L328N/I332E, L328Q/I332E, L328V/I332E, L328T/I332E, L328H/I332E, L328I/I332E, L328A, I332T, I332H, I332Y, I332A, S239E/V264I/I332E, S239Q/V264I/I332E, S239E/V264I/A330Y/I332E, S239E/V264I/S298A/A330Y/I332E, S239D/N297D/I332E, S239E/N297D/I332E, S239D/D265V/N297D/I332E, S239D/D265I/N297D/I332E, S239D/D265L/N297D/I332E, S239D/D265F/N297D/I332E, S239D/D265Y/N297D/I332E, S239D/D265H/N297D/I332E, S239D/D265T/N297D/I332E, V264E/N297D/I332E, Y296D/N297D/I332E, Y296E/N297D/I332E, Y296N/N297D/I332E, Y296Q/N297D/I332E, Y296H/N297D/I332E, Y296T/N297D/I332E, N297D/T299V/I332E, N297D/T299I/I332E, N297D/T299L/I332E, N297D/T299F/I332E, N297D/T299H/I332E, N297D/T299E/I332E, N297D/A330Y/I332E, N297D/S298A/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, AND S239D/264I/A330L/I332E, wherein the numbering of the residues in the Fc region is that of the EU index as in Kabat. See also PCT WO 2004/029207, Apr. 8, 2004, incorporated by reference herein.

Antibody-associated ADCC activity can be monitored and quantified through measurement of lactate dehydrogenase (LDH) release in the culture supernatant of cells expressing CS1 or any of the other antigens described herein (e.g., CD20, CD38, CD40, CD56, CD138, CD317 or KIR), which is rapidly released upon damage to the plasma membrane. The antigen-expressing cells are in certain embodiments myeloma cells, for example T-cell, NK-cell, or NKT cell myeloma cells. In some embodiments, the antigen-expressing cells are not myeloma cells, for example, normal NK cells. In various embodiments, the antibodies induce at least 10%, 20%, 30%, 40%, 50%, 60%, or 80% cytotoxicity of the target cells. An example of an ADCC assay that can be used to measure ADCC of an anti-CS1 antibody is that of Tai et al., 2008, Blood 112:1329-1337.

Also encompassed by the present disclosure is the use of scFv molecules that target myeloma cells or NK cells. The term “scFv” refers to a single chain Fv antibody in which the variable domains of the heavy chain and the light chain from a traditional antibody have been joined to form one chain.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

Complementary Determining Regions (“CDRs”) refers to the hypervariable regions in the light chain and the heavy chain variable domains. The more highly conserved portions of variable domains are called the framework regions (“FR”). The amino acid position/boundary delineating a hypervariable region of an antibody can vary, depending on the context and the various definitions known in the art. Some positions within a variable domain can be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The variable domains of native heavy and light chains each comprise four FR regions, largely by adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the target binding site of antibodies (See Kabat et al., Sequences of Proteins of Immunological Interest (National Institute of Health, Bethesda, Md. 1987)). As used herein, numbering of immunoglobulin amino acid residues is done according to the immunoglobulin amino acid residue numbering system of Kabat et al., unless otherwise indicated.

The term “antibody fragment” refers to a portion of a full-length antibody, generally the target binding or variable region. Examples of antibody fragments include Fab, Fab′, F(ab′)2 and Fv fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the VH and VL domains of an antibody, wherein these domains are present in a single polypeptide chain. Generally, the Fv polypeptide further comprises a polypeptide linker between the VH and VL domains which enables the sFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH-1 domain including one or more cysteines from the antibody hinge region. F(ab′) fragments are produced by cleavage of the disulfide bond at the hinge cysteines of the F(ab′)2 pepsin digestion product. Additional chemical couplings of antibody fragments are known to those of ordinary skill in the art.

In some embodiments, the antibodies described herein are monoclonal antibodies. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof. In other embodiments, including in vivo use of the antibodies in humans and in vitro detection assays, chimeric, primatized, humanized, or human antibodies can be used.

In some embodiments, the antibodies described herein are chimeric antibodies. The term “chimeric” antibody as used herein refers to an antibody having variable sequences derived from a non-human immunoglobulins, such as rat or mouse antibody, and human immunoglobulins constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties.

In some embodiments, the antibodies described herein are humanized antibodies. “Humanized” forms of non-human (e.g., murine) antibodies are chimeric immunoglobulins, immunoglobulin chains or fragments thereof (such as Fv, Fab, Fab′, F(ab′)2 or other target-binding subsequences of antibodies) which contain minimal sequences derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody can also comprise at least a portion of an immunoglobulin constant region (Fc), typically that of a human immunoglobulin template chosen. Humanization is a technique for making a chimeric antibody in which one or more amino acids or portions of the human variable domain have been substituted by the corresponding sequence from a non-human species. Humanized antibodies are antibody molecules generated in a non-human species that bind the desired antigen having one or more complementarity determining regions (CDRs) from the non-human species and framework (FR) regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Riechmann et al., 1988, Nature 332:323-7 and Queen et al., U.S. Pat. Nos. 5,530,101; 5,585,089; 5,693,761; 5,693,762; and 6,180,370 (each of which is incorporated by reference in its entirety). Antibodies can be humanized using a variety of techniques known in the art including, for example, CDR-grafting (EP239400; PCT publication WO 91/09967; U.S. Pat. Nos. 5,225,539; 5,530,101 and 5,585,089), veneering or resurfacing (EP592106; EP519596; Padlan (1991) Mol. Immunol. 28:489; Studnicka et al. (1994) Prot. Eng. 7:805; Roguska et al. (1994) Proc. Natl. Acad. Sci. 91:969), and chain shuffling (U.S. Pat. No. 5,565,332), all of which are hereby incorporated by reference in their entireties.

In some embodiments, humanized antibodies are prepared as described in Queen et al., U.S. Pat. Nos. 5,530,101; 5,585,089; 5,693,761; 5,693,762; and 6,180,370 (each of which is incorporated by reference in its entirety).

In some embodiments, the antibodies described herein are human antibodies. Completely “human” antibodies for use in the methods described herein can be desirable for therapeutic treatment of human patients. As used herein, “human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins, but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. In addition, companies such as Abgenix (Fremont, Calif.) (now part of Amgen) and Medarex (Princeton, N.J.) can be engaged to provide human antibodies directed against a selected antigen using technology similar to that described above. Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988, Biotechnology 12:899-903).

In some embodiments, the antibodies are primatized antibodies. The term “primatized antibody” refers to an antibody comprising monkey variable regions and human constant regions. Methods for producing primatized antibodies are known in the art. See e.g., U.S. Pat. Nos. 5,658,570; 5,681,722; and 5,693,780, which are incorporated herein by reference in their entireties.

In some embodiments, the antibodies are bispecific antibodies. Bispecific antibodies are monoclonal, preferably human or humanized, antibodies that have binding specificities for at least two different antigens. In the bispecific antibodies useful in the present methods, one of the binding specificities can be directed towards, e.g., CS1, the other can be for any other antigen (e.g., CD20), and preferably for a cell-surface protein, receptor, receptor subunit, tissue-specific antigen, virally derived protein, virally encoded envelope protein, bacterially derived protein, or bacterial surface protein, etc.

In some embodiments, the antibodies for use in the methods of the disclosure are derivatized antibodies. For example, but not by way of limitation, the derivatized antibodies that have been modified, e.g., by glycosylation, acetylation, pegylation, phosphorylation, amidation, derivatization by known protecting/blocking groups, proteolytic cleavage, linkage to a cellular ligand or other protein (see Section 4.3 for a discussion of antibody conjugates), etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

In some embodiments, the antibodies or fragments thereof can be antibodies or antibody fragments whose sequence has been modified to reduce at least one constant region-mediated biological effector function relative to the corresponding wild type sequence. To modify an antibody described herein such that it exhibits reduced binding to the Fc receptor, the immunoglobulin constant region segment of the antibody can be mutated at particular regions necessary for Fc receptor (FcγR) interactions (See e.g., Canfield and Morrison (1991) J. Exp. Med. 173:1483; and Lund et al. (1991) J. Immunol. 147:2657). Reduction in FcγR binding ability of the antibody can also reduce other effector functions which rely on FcγR interactions, such as opsonization and phagocytosis and antigen-dependent cellular cytotoxicity.

In yet other aspects, the antibodies or fragments thereof can be antibodies or antibody fragments that have been modified to acquire at least one constant region-mediated biological effector function relative to an unmodified antibody. To modify an antibody described herein, e.g., a myeloma cell targeting antibody, such that it exhibits increased binding to the Fcγ receptor (FcγR), the immunoglobulin constant region segment of the antibody can be mutated to enhance FcγR interactions (See, e.g., US Patent Publication No. 2006/0134709 A1). Enhancement of FcγR binding can increase antigen-dependent cellular cytotoxicity of an antibody described herein. In specific embodiments, an antibody described herein has a constant region that binds FcγRIIA, FcγRIIB and/or FcγRIIIA with greater affinity than the corresponding wild type constant region.

In yet other aspects, the antibodies described herein or fragments thereof can be antibodies or antibody fragments that have been modified to increase or reduce their binding affinities to the fetal Fc receptor, FcRn. To alter the binding affinity to FcRn, the immunoglobulin constant region segment of the antibody can be mutated at particular regions necessary for FcRn interactions (See e.g., PCT Publication No. WO 2005/123780). Increasing the binding affinity to FcRn should increase the antibody's serum half-life, and reducing the binding affinity to FcRn should conversely reduce the antibody's serum half-life. In particular embodiments, the antibody is of the IgG class in which at least one of amino acid residues 250, 314, and 428 of the heavy chain constant region is substituted with an amino acid residue different from that present in the unmodified antibody. The antibodies of IgG class include antibodies of IgG₁, IgG₂, IgG₃, and IgG₄. The substitution can be made at position 250, 314, or 428 alone, or in any combinations thereof, such as at positions 250 and 428, or at positions 250 and 314, or at positions 314 and 428, or at positions 250, 314, and 428, with positions 250 and 428 as a preferred combination. For each position, the substituting amino acid can be any amino acid residue different from that present in that position of the unmodified antibody. For position 250, the substituting amino acid residue can be any amino acid residue other than threonine, including, but not limited to, alanine, cysteine, aspartic acid, glutamic acid, phenylalanine, glycine, histidine, isoleucine, lysine, leucine, methionine, asparagine, proline, glutamine, arginine, serine, valine, tryptophan, or tyrosine. For position 314, the substituting amino acid residue can be any amino acid residue other than leucine, including, but not limited to, alanine, cysteine, aspartic acid, glutamic acid, phenylalanine, glycine, histidine, isoleucine, lysine, methionine, asparagine, proline, glutamine, arginine, serine, threonine, valine, tryptophan, or tyrosine. For position 428, the substituting amino acid residues can be any amino acid residue other than methionine, including, but not limited to, alanine, cysteine, aspartic acid, glutamic acid, phenylalanine, glycine, histidine, isoleucine, lysine, leucine, asparagine, proline, glutamine, arginine, serine, threonine, valine, tryptophan, or tyrosine. Specific combinations of suitable amino acid substitutions are identified in Table 1 of WO 2005/123780, which table is incorporated by reference herein in its entirety. See also, Hinton et al., U.S. Pat. Nos. 7,217,797, 7,361,740, 7,365,168, and 7,217,798, which are incorporated herein by reference in their entireties.

In yet other aspects, an antibody described herein has one or more amino acids inserted into one or more of its hypervariable region, for example as described in US 2007/0280931.

4.3 Antibody Conjugates

In some embodiments, the antibodies described herein are antibody conjugates that are modified, e.g., by the covalent attachment of any type of molecule to the antibody, such that covalent attachment does not interfere with binding to the antigen (e.g, CS1).

For example, in some embodiments an antibody can be conjugated to an effector moiety or a label. The term “effector moiety” as used herein includes, for example, antineoplastic agents, drugs, toxins, biologically active proteins, for example enzymes, other antibody or antibody fragments, synthetic or naturally occurring polymers, nucleic acids (e.g., DNA and RNA), radionuclides, particularly radioiodide, radioisotopes, chelated metals, nanoparticles and reporter groups such as fluorescent compounds or compounds which can be detected by NMR or ESR spectroscopy.

By way of another example, antibodies such as those targeted to myeloma cells described herein can be conjugated to an effector moiety, such as a cytotoxic agent, a radionuclide or drug moiety to modify a given biological response. The effector moiety can be a protein or polypeptide, such as, for example and without limitation, a toxin (such as abrin, ricin A, Pseudomonas exotoxin, or Diphtheria toxin), a signaling molecule (such as α-interferon, β-interferon, nerve growth factor, platelet derived growth factor or tissue plasminogen activator), a thrombotic agent or an anti-angiogenic agent (e.g., angiostatin or endostatin) or a biological response modifier such as a cytokine or growth factor (e.g., interleukin-1 (IL-1), interleukin-2 (IL-2), interleukin-6 (IL-6), granulocyte macrophage colony stimulating factor (GM-CSF), granulocyte colony stimulating factor (G-CSF), or nerve growth factor (NGF)).

In another example the effector moieties can be cytotoxins or cytotoxic agents. Examples of cytotoxins and cytotoxic agents include taxol, cytochalasin B, gramicidin D, ethidium bromide, emetine, mitomycin, etoposide, tenoposide, vincristine, vinblastine, colchicin, doxorubicin, daunorabicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

Effector moieties also include, but are not limited to, antimetabolites (e.g. methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C5 and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, anthramycin (AMC), calicheamicins or duocarmycins), and anti-mitotic agents (e.g., vincristine and vinblastine).

Other effector moieties can include radionuclides such as, but not limited to, In111 and Y90, Lu177, Bismuth213, Californium252, Iridium192 and Tungsten18s/Rhenium188 and drugs such as, but not limited to, alkylphosphocholines, topoisomerase I inhibitors, taxoids and suramin.

Techniques for conjugating such effector moieties to antibodies are well known in the art (See, e.g., Hellstrom et al., Controlled Drug Delivery, 2nd Ed., at pp. 623-53 (Robinson et al., eds., 1987); Thorpe et al. (1982) Immunol. Rev. 62:119 and Dubowchik et al. (1999) Pharmacology and Therapeutics 83:67).

In one example, the antibody or fragment thereof is fused via a covalent bond (e.g., a peptide bond), at optionally the N-terminus or the C-terminus, to an amino acid sequence of another protein (or portion thereof; preferably at least a 10, 20 or 50 amino acid portion of the protein). Preferably the antibody, or fragment thereof, is linked to the other protein at the N-terminus of the constant domain of the antibody. Recombinant DNA procedures can be used to create such fusions, for example as described in WO 86/01533 and EP0392745. In another example the effector molecule can increase half-life in vivo, and/or enhance the delivery of an antibody across an epithelial barrier to the immune system. Examples of suitable effector molecules of this type include polymers, albumin, albumin binding proteins or albumin binding compounds such as those described in WO 2005/117984.

In some embodiments, the antibodies described herein can be attached to poly(ethyleneglycol) (PEG) moieties. For example, if the antibody is an antibody fragment, the PEG moieties can be attached through any available amino acid side-chain or terminal amino acid functional group located in the antibody fragment, for example any free amino, imino, thiol, hydroxyl or carboxyl group. Such amino acids can occur naturally in the antibody fragment or can be engineered into the fragment using recombinant DNA methods. See for example U.S. Pat. No. 5,219,996. Multiple sites can be used to attach two or more PEG molecules. Preferably PEG moieties are covalently linked through a thiol group of at least one cysteine residue located in the antibody fragment. Where a thiol group is used as the point of attachment, appropriately activated effector moieties, for example thiol selective derivatives such as maleimides and cysteine derivatives, can be used.

In another example, an antibody conjugate is a modified Fab′ fragment which is PEGylated, i.e., has PEG (poly(ethyleneglycol)) covalently attached thereto, e.g., according to the method disclosed in EP0948544. See also Poly(ethyleneglycol) Chemistry, Biotechnical and Biomedical Applications, (J. Milton Harris (ed.), Plenum Press, New York, 1992); Poly(ethyleneglycol) Chemistry and Biological Applications, (J. Milton Harris and S. Zalipsky, eds., American Chemical Society, Washington D.C., 1997); and Bioconjugation Protein Coupling Techniques for the Biomedical Sciences, (M. Aslam and A. Dent, eds., Grove Publishers, New York, 1998); and Chapman (2002) Advanced Drug Delivery Reviews 54:531.

The word “label” when used herein refers to a detectable compound or composition which can be conjugated directly or indirectly to an antibody described herein. The label can itself be detectable (e.g., radioisotope labels or fluorescent labels) or, in the case of an enzymatic label, can catalyze chemical alteration of a substrate compound or composition which is detectable. Useful fluorescent moieties include, but are not limited to, fluorescein, fluorescein isothiocyanate, rhodamine, 5-dimethylamine-1-napthalenesulfonyl chloride, phycoerythrin and the like. Useful enzymatic labels include, but are not limited to, alkaline phosphatase, horseradish peroxidase, glucose oxidase and the like.

4.4 Therapeutic Methods, Pharmaceutical Compositions and Routes of Administration

The combination of expanded and activated autologous NK cells and antibodies targeted to an antigen that is expressed on the surface of myeloma cells is useful for treating multiple myeloma according to the methods described herein. In some aspects, the combination of expanded and activated autologous NK cells and an antibody that targets the KIR protein that is expressed on the surface of NK cells is useful for treating multiple myeloma according to the methods described herein. In other aspects, the combination of expanded and activated autologous NK cells, an antibody that targets myeloma cells and an antibody that targets the KIR protein on NK cells is useful for treating multiple myeloma.

In specific embodiments, an antibody targeted to myeloma cells and/or an antibody targeted to NK cells is administered prior to administration of the autologous NK cells. In certain embodiments, the antibody is administered 0 to 60 days prior to the administration of the expanded autologous NK cells. In some embodiments, an antibody is administered from about 30 minutes to about 1 hour prior to the administration of NK cells, or from about 1 hour to about 2 hours, or from about 2 hours to about 4 hours, or from about 4 hours to about 6 hours, or from about 6 hours to about 8 hours, or from about 8 hours to about 16 hours, or from about 16 hours to 1 day, or from about 1 to 5 days, or from about 5 to 10 days, or from about 10 to 15 days, or from about 15 to 20 days, or from about 20 to 30 days, or from about 30 to 40 days, and from about 40 to 50 days, or from about 50 to 60 days prior to the administration of the autologous NK cells. In certain embodiments, the antibody is an anti-CS1 antibody such as elotuzumab.

In still other embodiments, the antibody is administered subsequent to administration of the expanded autologous NK cells. In some embodiments, the antibody is administered from about 30 minutes to about 1 hour subsequent to the administration of NK cells, or from about 1 hour to about 2 hours, or from about 2 hours to about 4 hours, or from about 4 hours to about 6 hours, or from about 6 hours to about 8 hours, or from about 8 hours to about 16 hours, or from about 16 hours to 1 day, or from about 1 to 5 days, or from about 5 to 10 days, or from about 10 to 15 days, or from about 15 to 20 days, or from about 20 to 30 days, or from about 30 to 40 days, and from about 40 to 50 days, or from about 50 to 60 days subsequent to the administration of the autologous NK cells. In certain embodiments, the antibody is an anti-CS1 antibody such as elotuzumab.

In other embodiments, an antibody targeting myeloma cells and/or an antibody targeting NK cells is administered concurrently with the autologous NK cells. In certain specific embodiments, the antibody is the anti-CS1 antibody elotuzumab and is administered prior to administration of the autologous NK cells.

Expanded autologous NK cells for use in combination with an antibody as described herein are typically administered to a patient by intravenous injection or infusion. In certain embodiments, NK cells are derived from PBMCs obtained from the patient by apheresis. NK cells are expanded as described above, collected from the culture medium, washed, and suspended in a physiologically compatible carrier for injection into the patient. As used herein, the term “physiologically compatible carrier” refers to a carrier that is compatible with the other ingredients of the formulation and not deleterious to the recipient thereof. Physiologically compatible carriers are known to those of skill in the art. Examples of suitable carriers include phosphate buffered saline, Hank's balanced salt solution +/−glucose (HBSS), Ringer's solution, dextrose solution, and a solution of 5% human serum albumin in 0.9% sodium chloride for injection. In other embodiments, PBMCs are obtained from the patient, cryopreserved and thawed before NK cell expansion as described above. In some embodiments, expanded NK cells are depleted of residual T-cells by methods known in the art, e.g., using the CliniMACS System (Miltenyi) for cell selection, before administration to the patient.

In typical embodiments, an effective dose of autologous NK cells to be administered to a subject with multiple myeloma in combination with an antibody described herein is about 1×10⁵ cells/kg of body weight, such as about 5×10⁵ cells/kg of body weight, such as about 1×10⁶ cells/kg of body weight, such as about 5×10⁶ cells/kg of body weight, such as about 1×10⁷ cells/kg of body weight, such as about 2×10⁷ cells/kg of body weight, such as about 3×10⁷ cells/kg of body weight, such as about 4×10⁷ cells/kg of body weight, such as about 5×10⁷ cells/kg of body weight, such as about 7.5×10⁷ cells/kg of body weight or such as about 1×10⁸ cells/kg of body weight. In certain embodiments an effective dose of autologous NK cells for treatment of multiple myeloma ranges between any two of the foregoing values, such as from about 1×10⁷ to about 1×10⁸ cells/kg of body weight, etc.

In certain embodiments, the dose of autologous NK cells to be administered to a subject with multiple myeloma contains less than about 1×10⁵ T-cells/kg of body weight, such as less than about 5×10⁴ T-cells/kg of body weight, such as less than about 1×10⁴ T-cells/kg of body weight, such as less than about 5×10³T-cells/kg of body weight, such as less than about 1×10³ T-cells/kg of body weight. In certain embodiments the dose of autologous NK cells for treatment of multiple myeloma contains an amount of T-cells ranging between any two of the foregoing values, such as from less than about 1×10⁵ to less than about 1×10³ T-cells/kg of body weight, etc.

The effective dose of autologous NK cells can be administered in a single dose or in multiple doses. In certain embodiments, the effective dose of autologous NK cells is administered in a single dose by continuous intravenous administration. In certain embodiments, expanded NK cells are administered over a period of time from about 1 to about 24 hours, such as over a period of about 1 to 2 hours. Dosages can be repeated from about 1 to about 4 weeks or more, for a total of 4 or more doses. Typically, dosages are repeated once every week, once every two weeks, or once a month for a minimum of 4 doses to a maximum of 52 doses.

Determination of the effective dosage, total number of doses, and length of treatment with autologous expanded NK cells in combination with an antibody that targets myeloma cells and/or an antibody that targets the KIR protein on NK cells is well within the capabilities of those skilled in the art, and can be determined using a standard dose escalation study to identify the maximum tolerated dose (MTD) (see, e.g., Miller et al. (2005) Blood 105:3051; Richardson et al. (2002) Blood, 100(9):3063, the contents of which is incorporated herein by reference).

The antibodies described herein for use in combination with autologous NK cells can be administered to a patient by a variety of routes such as orally, transdermally, subcutaneously, intranasally, intravenously, intramuscularly, intrathecally, topically or locally. The most suitable route for administration in any given case will depend on the particular antibody, the subject, and the nature and severity of the disease and the physical condition of the subject. Typically, an anti-CS1 antibody such as elotuzumab will be administered intravenously.

In typical embodiments, an antibody that targets myeloma cells and/or an antibody that targets the KIR protein of NK cells is present in a pharmaceutical composition at a concentration sufficient to permit intravenous administration at 0.5 mg/kg to 20 mg/kg. In some embodiments, the concentration of elotuzumab suitable for use in the compositions and methods described herein includes, but is not limited to, 0.5 mg/kg, 0.75 mg/kg, 1 mg/kg, 2 mg/kg, 2.5 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or a concentration ranging between any of the foregoing values, e.g., 1 mg/kg to 10 mg/kg, 5 mg/kg to 15 mg/kg, or mg/kg to 18 mg/kg.

The effective dose of an antibody described herein can range from about 0.001 to about 750 mg/kg per single (e.g., bolus) administration, multiple administrations or continuous administration, or to achieve a serum concentration of 0.01-5000 μg/ml serum concentration per single (e.g., bolus) administration, multiple administrations or continuous administration, or any effective range or value therein depending on the route of administration and the age, weight and condition of the subject. In certain embodiments, each dose can range from about 0.5 mg to about 50 mg per kilogram of body weight or from about 3 mg to about 30 mg per kilogram body weight. The antibody can be formulated as an aqueous solution.

Pharmaceutical compositions can be conveniently presented in unit dose forms containing a predetermined amount of an antibody described herein per dose. Such a unit can contain 0.5 mg to 5 g, for example, but without limitation, 1 mg, 10 mg, 20 mg, 30 mg, 40 mg, 50 mg, 100 mg, 200 mg, 300 mg, 400 mg, 500 mg, 750 mg, 1000 mg, or any range between any two of the foregoing values, for example 10 mg to 1000 mg, 20 mg to 50 mg, or 30 mg to 300 mg. Pharmaceutically acceptable carriers can take a wide variety of forms depending, e.g., on the condition to be treated or route of administration.

The effective dose of the antibody can be administered in a single dose or in multiple doses. In certain embodiments, the effective dose of the antibody is administered in a single dose by continuous intravenous administration. In certain embodiments, the antibody is administered over a period of time from about 1 to about 24 hours, such as over a period of about 1 to 2 hours. Dosages can be repeated from about 1 to about 4 weeks or more, for a total of 4 or more doses. Typically, dosages are repeated once every week, once every two weeks, or once a month for a minimum of 4 doses to a maximum of 52 doses.

Determination of the effective dosage, total number of doses, and length of treatment with an antibody that targets myeloma cells and/or with an antibody that targets the NK cell KIR protein is well within the capabilities of those skilled in the art, and can be determined using a standard dose escalation study to identify the maximum tolerated dose (MTD) (see, e.g., Richardson et al. (2002) Blood, 100(9):3063, the content of which is incorporated herein by reference).

Therapeutic formulations of the antibodies suitable for the methods described herein can be prepared for storage as lyophilized formulations or aqueous solutions by mixing the antibody having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives must be nontoxic to the recipients at the dosages and concentrations employed.

Buffering agents help to maintain the pH in the range which approximates physiological conditions. They can present at concentration ranging from about 2 mM to about 50 mM. Suitable buffering agents include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives can be added to retard microbial growth, and can be added in amounts ranging from 0.2%-1% (w/v). Suitable preservatives include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions and include polhydric sugar alcohols, preferably trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran. Stabilizers can be present in the range from 0.1 to 10,000 weights per part of weight active protein.

Non-ionic surfactants or detergents (also known as “wetting agents”) can be added to help solubilize the therapeutic agent as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), Pluronic polyols, polyoxyethylene sorbitan monoethers (TWEEN®-20, TWEEN®-80, etc.). Non-ionic surfactants can be present in a range of about 0.05 mg/ml to about 1.0 mg/ml, or in a range of about 0.07 mg/ml to about 0.2 mg/ml.

Additional miscellaneous excipients include bulking agents (e.g., starch), chelating agents (e.g., EDTA), antioxidants (e.g., ascorbic acid, methionine, vitamin E), and cosolvents.

The antibody formulation herein can also contain a second therapeutic agent in addition to a myeloma cell targeting antibody and/or an NK cell targeting antibody. Examples of suitable second therapeutic agents are provided in Section 4.5 below.

In certain embodiments, a unit dose of antibody is administered before, after or concurrently with a unit dose of autologous NK cells. In other embodiments, the ratio of dosing of the antibody to dosing of the autologous NK cells is 2:1, or 3:1 or 4:1, or 5:1 or more, i.e., for every one dose of autologous NK cells, the patient receives 2, 3, 4, etc. doses of the antibody. In still other embodiments, a first dose of the antibody is administered prior to administration of the autologous NK cells, and additional doses (which can be of the same or different magnitude as the first dose, and which can be the same antibody as used in the pretreatment or a different antibody targeted to the same antigen or to a different antigen expressed on myeloma cells or an antibody targeted to the NK cell KIR protein) are administered subsequent to NK cell administration as a maintenance therapy. In yet further embodiments, additional doses of the antibody can be used as a salvage therapy. In various embodiments, additional doses of the antibody can be used as a maintenance therapy, either alone or in combination with one or more therapeutic agents described in Section 4.5 below.

It will be recognized by one of skill in the art that the optimal quantity and spacing of individual dosages of autologous NK cells and of an antibody that targets myeloma cells and/or an antibody that targets NK cells will be determined by the nature and extent of the multiple myeloma being treated, the form, route and site of administration, the age and physical condition of the particular subject being treated, the particular antibody, and the therapeutic regimen (e.g., whether an additional therapeutic agent is used), and that the skilled artisan will readily determine the appropriate dosages and dosing schedules to be used. The dosages can be repeated as often as appropriate. If side effects develop the amount and/or frequency of the dosages can be altered or reduced, in accordance with normal clinical practice.

4.5 Combination with Other Treatment Strategies or Agents

In various embodiments, the administration of autologous NK cells in a combination therapy with an antibody that targets myeloma cells and/or an antibody that targets the NK cell KIR protein is combined with another treatment strategy. In some embodiments, the combination therapy can be administered prior to the initiation of a treatment regimen incorporating stem cell transplantation. In other embodiments, the combination therapy can be administered following a treatment regimen incorporating stem cell transplantation. The stem cell transplantation regimen can be autologous or syngeneic, tandem autologous, “mini” allogeneic, and/or combinations thereof.

In still other embodiments, the combination therapy can be administered prior to delayed rescue with stem cells.

In some embodiments, the combination of autologous NK cells and an antibody that targets myeloma cells and/or an antibody that targets NK cells is administered before or after non-myeloablative chemotherapy with, e.g., low doses of cyclophosphamide and fludarabine or low-dose radiation.

In other embodiments, the combination of autologous NK cells and an antibody that targets myeloma cells and/or an antibody that targets NK cells is administered after conditioning therapy, such as conditioning therapy with cyclophosphamide and fludarabine or melphalan and fludarabine.

In certain embodiments, administration of the combination of autologous NK cell and an antibody described herein can precede or follow administration of an additional therapeutic agent. As a non-limiting example, the combination therapy and the additional therapeutic agent can be administered concurrently for a period of time, followed by a second period of time in which the administration of the combination therapy and the additional therapeutic agent is alternated. In certain embodiments, the additional therapeutic agent can be administered concurrently with the antibody and, in some embodiments, in the same pharmaceutical composition.

Because of the potentially synergistic effects of administering the combination therapy and the additional therapeutic agent, such agents can be administered in amounts that, if any of the agents is administered alone, is/are not therapeutically effective. For example, in various embodiments, the dosage of the combination therapy and/or the dosage of the additional therapeutic agent administered is about 10% to 90% of the generally accepted efficacious dose range for either the combination therapy or the additional agent therapy alone. In some embodiments, about 10%, about 15%, about 25%, about 30%, about 40%, about 50%, about 60%, about 75%, or about 90% of the generally accepted efficacious dose range is used, or a dosage ranging between any of the foregoing values (e.g., 10% to 40%, 30% to 75%, or 60% to 90% of the of the generally accepted efficacious dose range) is used.

Therapeutic agents that can be used in combination with the antibodies described herein include, but are not limited to, targeted agents, conventional chemotherapy agents, hormonal therapy agents, and supportive care agents. One or more therapeutic agents from the different classes, e.g., targeted, conventional chemotherapeutic, hormonal, and supportive care, and/or subclasses can be combined in the compositions described herein. The various classes described herein can be further divided into subclasses. By way of example, targeted agents can be separated into a number of different subclasses depending on their mechanism of action. As will be apparent to those of skill in the art, the agents can have more than one mechanism of action, and thus, could be classified into one or more subclasses. For purposes of the compositions and methods described herein, the following subclasses have been identified: anti-angiogenic, inhibitors of growth factor signaling, immunomodulators, inhibitors of protein synthesis, folding and/or degradation, inhibitors of gene expression, pro-apoptotic agents, agents that inhibit signal transduction and agents with “other” mechanisms of action. Typically, the mechanism of action for agents falling into the “other” subclass is unknown or poorly characterized.

For example, in some embodiments, targeted agents, such as bevacizumab, sutinib, sorafenib, 2-methoxyestradiol or 2ME2, finasunate, PTK787, vandetanib, aflibercept, volociximab, etaracizumab (MEDI-522), cilengitide, erlotinib, cetuximab, panitumumab, gefitinib, trastuzumab, TKI258, CP-751,871, atacicept, rituximab, alemtuzumab, aldesleukine, atlizumab, tocilizumab, temsirolimus, everolimus, NPI-1387, MLNM3897, HCD122, SGN-40, HLL1, huN901-DM1, atiprimod, natalizumab, bortezomib, carfilzomib, NPI-0052, tanespimycin, saquinavir mesylate, ritonavir, nelfinavir mesylate, indinavir sulfate, belinostat, LBH589, mapatumumab, lexatumumab, AMG951, ABT-737, oblimersen, plitidepsin, SCIO-469, P276-00, enzastaurin, tipifamib, perifosine, imatinib, dasatinib, lenalidomide, thalidomide, simvastatin, and celecoxib can be combined with an anti-CS1 antibody, such as elotuzumab, and/or with an antibody that targets the KIR protein of NK cells and used to treat MM patients.

By way of another example, conventional chemotherapy agents, such as alklyating agents (e.g., oxaliplatin, carboplatin, cisplatin, cyclophosphamide, melphalan, ifosfamide, uramustine, chlorambucil, carmustine, mechloethamine, thiotepa, busulfan, temozolomide, dacarbazine), anti-metabolic agents (e.g., gemcitabine, cytosine arabinoside, Ara-C, capecitabine, 5FU (5-fluorouracil), azathioprine, mercaptopurine (6-MP), 6-thioguanine, aminopterin, pemetrexed, methotrexate), plant alkaloid and terpenoids (e.g., docetaxel, paclitaxel, vincristine, vinblastin, vinorelbine, vindesine, etoposide, VP-16, teniposide, irinotecan, topotecan), anti-tumor antibiotics (e.g., dactinomycin, doxorubicin, liposomal doxorubicin, daunorubicin, daunomycin, epirubicin, mitoxantrone, adriamycin, bleomycin, plicamycin, mitomycin C, caminomycin, esperamicins), and other agents (e.g., darinaparsin) can be combined with an anti-CS1 antibody, such as elotuzumab and/or with an antibody that targets the KIR protein of NK cells and used to treat MM.

By way of another example, hormonal agents such as anastrozole, letrozole, goserelin, tamoxifen, dexamethasone, prednisone, and prednisilone can be combined with an anti-CS1 antibody, such as elotuzumab and/or an antibody that targets the KIR protein of NK cells and used to treat MM.

By way of another example, supportive care agents such as pamidronate, zoledonic acid, ibandronate, gallium nitrate, denosumab, darbepotin alpha, epoetin alpha, eltrombopag, and pegfilgrastim can be combined with an anti-CS1 antibody, such as elotuzumab and/or an antibody that targets the KIR protein of NK cells and used to treat MM.

The therapeutic agents can be administered in any manner found appropriate by a clinician and are typically provided in generally accepted efficacious dose ranges, such as those described in the Physician Desk Reference, 56th Ed. (2002), Publisher Medical Economics, New Jersey. In other embodiments, a standard dose escalation study can be performed to identify the maximum tolerated dose (MTD) (see, e.g., Richardson, et al. 2002, Blood, 100(9):3063-3067, the content of which is incorporated herein by reference).

In some embodiments, doses less than the generally accepted efficacious dose of a therapeutic agent can be used. For example, in various embodiments, the composition comprises a dosage that is less than about 10% to 75% of the generally accepted efficacious dose range. In some embodiments, at least about 10% or less of the generally accepted efficacious dose range is used, at least about 15% or less, at least about 25%, at least about 30% or less, at least about 40% or less, at least about 50% or less, at least about 60% or less, at least about 75% or less, and at least about 90%.

The therapeutic agents can be administered singly or sequentially, or in a cocktail with other therapeutic agents, as described below. The therapeutic agents can be administered orally, intravenously, systemically by injection intramuscularly, subcutaneously, intrathecally or intraperitoneally.

In some embodiments, the therapeutic agents provided in the pharmaceutical composition(s) are selected from the group consisting of dexamethasone, thalidomide, pomalidomide (Actimid™), vincristine, carmustine (BCNU), melphalan, cyclophosphamide, prednisone, doxorubicin, cisplatin, etoposide, bortezomib (Velcade®), lenalidomide (Revlimid®), ara-C, and/or combinations thereof.

In certain embodiments, the combination therapy is administered with a corticosteroid in order to prevent infusion reactions that can result from administration of one or more antibodies described herein. Accordingly, in certain embodiments, the corticosteroid is administered prior to administration of the antibody. In other embodiments, the corticosteroid is administered concurrently with administration of the antibody. In still other embodiments, the corticosteroid is administered subsequent to administration of the antibody. In various embodiments, the corticosteroid is selected from prednisone, methylprednisone, betamethasone, budesonide, dexamethasone, and hydrocortisone. In certain embodiments, the steroid is administered intravenously prior to administration of the antibody. In particular embodiments, the corticosteroid is methylprednisone. In some embodiments, an antihistamine is administered concurrently with the corticosteroid. Suitable antihistamines include, but are not limited to, chlophenamine, alizapride, cetirizine, clemastine, promethazine, dexchlorpheniramine, diphenhydramine and dimentindene.

In certain embodiments, the combination therapy is administered with a cytokine. In some embodiments, the cytokine is selected from IL-2, IL-4, IL-7, IL-12 and IL-15.

In various embodiments, the combination therapy is administered with an additional antibody targeted to an antigen other than the antigen to which the first antibody is targeted, such as CD3.

Administration of one or more of the additional therapeutic agents described herein can be by any means known in the art, including, but not limited to, oral, rectal, nasal, topical (including buccal and sublingual) or parenteral (including subcutaneous, intramuscular, intravenous and intradermal) administration and will depend in part, on the available dosage form. For example, therapeutic agents that are available in a pill or capsule format typically are administered orally. However, oral administration generally requires administration of a higher dose than does intravenous administration. Determination of the optimal route of administration for a particular subject is well within the capabilities of those skilled in the art, and in part, will depend on the dose needed versus the number of times per month administration is required.

4.6 Effectiveness of Treatment Regimens

The use of expanded autologous NK cells in combination with an antibody targeted to myeloma cells (e.g., elotuzumab) and/or an antibody that targets the KIR protein on NK cells can be used to develop an effective treatment strategy based on the stage of myeloma being treated (see, e.g., Multiple Myeloma Research Foundation, Multiple Myeloma Stem Cell Transplantation 1-30 (2004); U.S. Pat. Nos. 6,143,292, and 5,928,639, Igarashi et al. (2004) Blood 104(1): 170, Maloney et al. (2003) Blood 102(9):3447, Badros et al. (2002) J Clin Oncol. 20:1295, Tricot, et al. (1996), Blood 87(3):1196, the contents of which are incorporated herein by reference).

The staging system most widely used since 1975 is the Durie-Salmon system, in which the clinical stage of disease (Stage I, II, or III) is based on four measurements (see, e.g., Durie et al. (1975) Cancer, 36:842). These four measurements are: (1) levels of monoclonal (M) protein (also known as paraprotein) in the patient's serum and/or the urine; (2) the number of lytic bone lesions; (3) hemoglobin values; and, (4) serum calcium levels. The three stages can be further divided according to renal function, classified as A (relatively normal renal function, serum creatinine value<2.0 mg/dL) and B (abnormal renal function, creatinine value>2.0 mg/dL). A new, simpler alternative is the International Staging System (ISS) (see, e.g., Greipp et al., 2003, “Development of an international prognostic index (IPI) for myeloma: report of the international myeloma working group”, The Hematology). The ISS is based on the assessment of two blood test results, beta2-microglobulin and albumin, which categorizes patients into three prognostic groups irrespective of the type of therapy.

Treatment of multiple myeloma patients using the methods described herein typically elicits a beneficial response as defined by the European Group for Blood and Marrow transplantation (EBMT). Table 2 lists the EBMT criteria for response.

TABLE 2
EBMT/IBMTR/ABMTR¹ Criteria for Response

| | |
|---|---|
| Complete Response | No M-protein detected in serum or urine by |
| | immunofixation for a minimum of 6 weeks and |
| | fewer than 5% plasma cells in bone marrow |
| Partial Response | >50% reduction in serum M-protein level |
| | and/or 90% reduction in urine free light chain |
| | excretion or reduction to <200 mg/24 hrs for 6 |
| | weeks² |
| Minimal Response | 25-49% reduction in serum M-protein level |
| | and/or 50-89% reduction in urine free light |
| | chain excretion which still exceeds 200 mg/24 hrs |
| | for 6 weeks³ |
| No Change | Not meeting the criteria or either minimal |
| | response or progressive disease |
| Plateau | No evidence of continuing myeloma-related |
| | organ or tissue damage, <25% change in M- |
| | protein levels and light chain excretion for 3 |
| | months |
| Progressive Disease | Myeloma-related organ or tissue damage |
| | continuing despite therapy or its reappearance |
| | in plateau phase, >25% increase in serum M- |
| | protein level (>5 g/L) and/or >25% increase in |
| | urine M-protein level (>200 mg/24 hrs) and/or |
| | >25% increase in bone marrow plasma cells (at |
| | least 10% in absolute terms)² |
| Relapse | Reappearance of disease in patients previously |
| | in complete response, including detection of |
| | paraprotein by immunofixation |

¹EBMT: European Group for Blood and Marrow transplantation; IBMTR: International Bone Marrow Transplant Registry; ABMTR: Autologous Blood and Marrow Transplant Registry.
²For patients with non-secretory myeloma only, reduction of plasma cells in the bone marrow by >50% of initial number (partial response) or 25-49% of initial number (minimal response) is required.
³In non-secretory myeloma, bone marrow plasma cells should increase by >25% and at least 10% in absolute terms; MRI examination may be helpful in selected patients.

Additional criteria that can be used to measure the outcome of a treatment include “near complete response” and “very good partial response”. A “near complete response” is defined as the criteria for a “complete response” (CR), but with a positive immunofixation test. A “very good partial response” is defined as a greater than 90% decrease in M protein (see, e.g., Multiple Myeloma Research Foundation, Multiple Myeloma: Treatment Overview 9 (2005)).

The response of an individual clinically manifesting at least one symptom associated with multiple myeloma to the methods described herein depends in part, on the severity of disease, e.g., Stage I, II, or III, and in part, on whether the patient is newly diagnosed or has late stage refractory multiple myeloma. Thus, in some embodiments, treatment with a combination of autologous activated NK cells and an antibody such as elotuzumab that targets myeloma cells and/or an antibody that targets NK cells elicits a complete response.

In other embodiments, treatment with a combination of autologous activated NK cells and an antibody such as elotuzumab that targets myeloma cells and/or an antibody that targets NK cells elicits a very good partial response or a partial response.

In various embodiments, treatment with a combination of autologous activated NK cells and an antibody such as elotuzumab that targets myeloma cells and/or an antibody that targets NK cells elicits a minimal response.

In other embodiments, treatment with a combination of autologous activated NK cells and an antibody such as elotuzumab that targets myeloma cells and/or an antibody that targets NK cells prevents the disease from progressing, resulting in a response classified as “no change” or “plateau” by the EBMT.

### 5. EXAMPLE 1

### Ex Vivo Expansion and Characterization of NK Cells from Multiple Myeloma Patients

5.1 Methods

Peripheral blood mononuclear cells (PMBC) from 8 patients with multiple myeloma were collected from blood samples by centrifugation on a Lymphoprep density step (Nycomed, Oslo, Norway), and were washed twice with unsupplemented RPMI medium and resuspended.

PMBC (1.5×10⁶) were incubated in a 24-well tissue culture plate for 14 days with 10⁶ irradiated K562 cells transfected with 4-1BBL ligand and membrane-bound IL-15 (K562-mb15-41BBL cells) in the presence of 300 U/ml of IL-2 in RPMI-1640 and 10% FCS. Medium was exchanged every 2 days with fresh medium and IL-2. After 7 days of co-culture, cells were restimulated by addition of 10⁶ irradiated modified K562 cells. The growth of NK cells, T cells and NKT cells in co-culture with K562-mb15-41BBL cells during the 14-day period was monitored by flow cytometry.

After expansion, cells were harvested and labeled with anti-CD3 fluorescein isothiocyanate (FITC) and anti-CD56 phycoerythrin (PE) antibodies. Non-expanded NK cells from the same patients were also labeled with the antibodies. Antibody staining of non-expanded and expanded NK cells was detected with a FACScan flow cytometer (Becton Dickinson). (See Imai et al. (2004) Leukemia 18:676; Ito et al. (1999) Blood 93:315; Srivannaboon et al. (2001) Blood 97:752).

NK cells were characterized by immunophenotyping using antibodies to the following molecules: NKp30, NKp44, NKp46, NK-p80, NKG2D and CD16 as described in Shi et al. (1008) Blood 111:1309.

5.2 Results

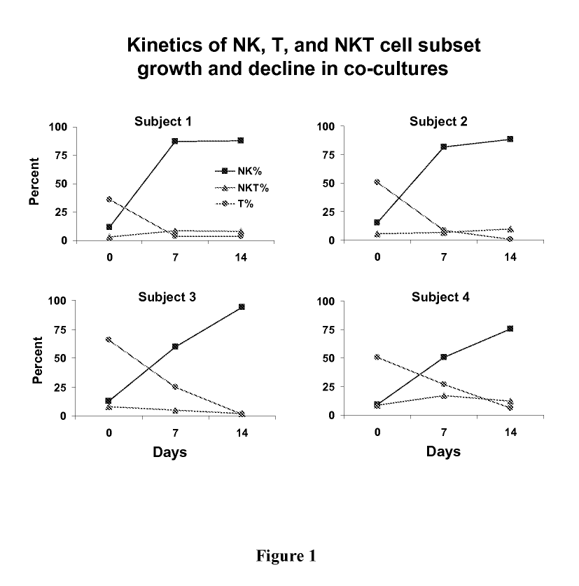

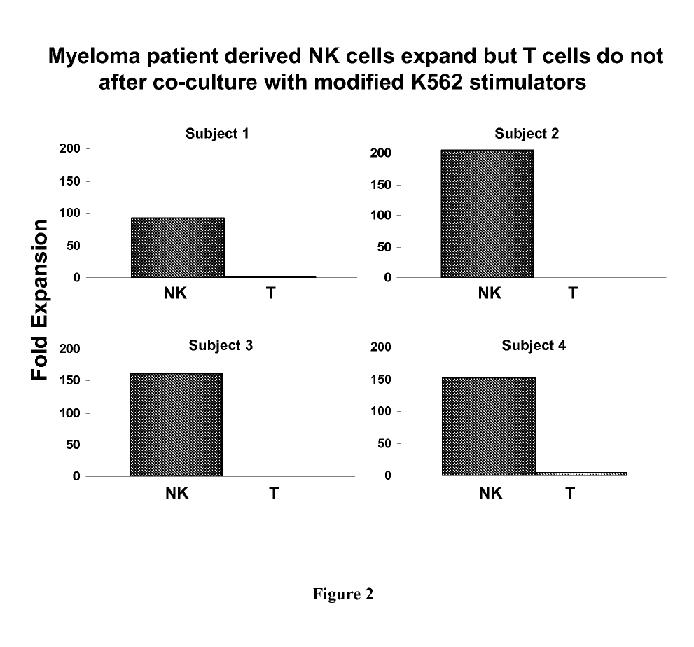

Over the 14-day culturing period, the number of NK cells expanded to account for over 75% of total cells in most of the ex vivo cultures, while the number of T-cells declined from around 25-50% to less than 10% of total cells. The number of NKT cells remained at a similarly low level (less than 10% of total cells) in all subjects. (FIG. 1) NK cells from four of the eight subjects showed significant expansion after 14 days of culturing (from 92-204-fold; average expansion 152-fold), while the number of T-cells in the ex vivo cultures did not expand. (FIG. 2)

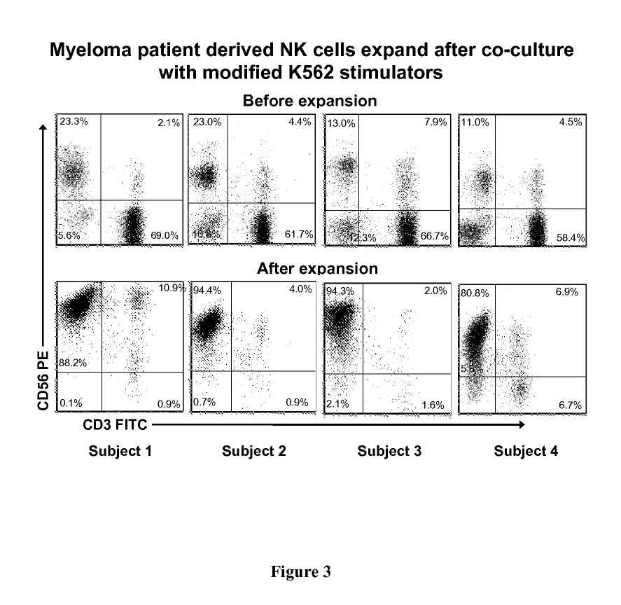

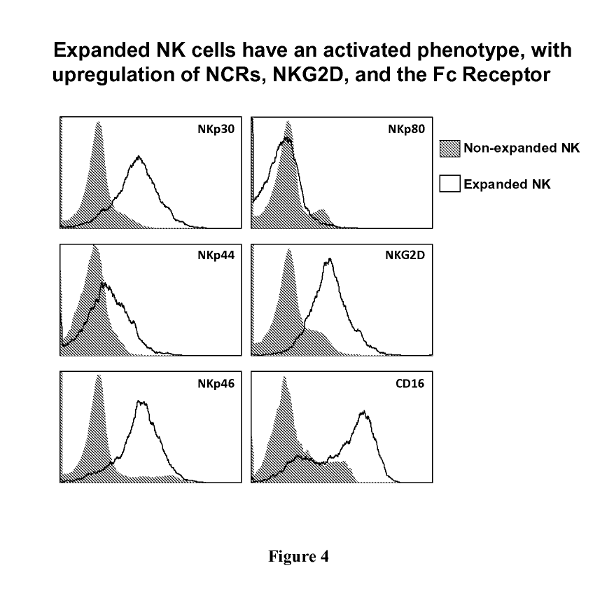

Non-expanded NK cells exhibited high expression of CD3 and low expression of CD65 on the cell surface. After expansion in the presence of modified K562 cells, NK cells showed high expression of CD65 and low expression of CD3. Expanded cells lacked T-cell receptors. (FIG. 3). Expanded NK cells from myeloma patients were found to express the NK-cell activating receptor NKG2D and natural cytotoxicity receptors NKp30, NKp44, and NKp46, indicating that the expanded NK cells are activated. (FIG. 4)

### 6. EXAMPLE 2

### Lysis of Multiple Myeloma Cells by Ex Vivo Expanded Autologous NK Cells Alone or in Combination with Elotuzumab

6.1 Methods

Target cells for this assay included (i) autologous PHA blasts; (ii) autologous CD34⁺ cells; (iii) autologous multiple myeloma cells; and (iv) K562 cells. Multiple myeloma cells from each subject were divided into the following treatment batches: (1) for treatment with expanded NK cells alone; (2) for treatment with non-expanded NK cells alone; (3) for pretreatment with elotuzumab followed by treatment with expanded NK cells; (4) for pretreatment with elotuzumab followed by treatment with non-expanded NK cells; (5) for pretreatment with an isotype control antibody followed by treatment with expanded NK cells; and (6) for pretreatment with an isotype control antibody followed by treatment with non-expanded NK cells. Target cells were cultured in vitro as previously described. See Colonna et al. (1993) Science 260:1121. Batches of multiple myeloma cells were pretreated either with 10 μg/ml elotuzumab or 10 μg/ml of control antibody before the ⁵¹Cr assay.

Target cells were labeled and the ⁵¹Cr release assay was performed as described in Colonna et al. (1993) Science 260:1121.

6.2 Results

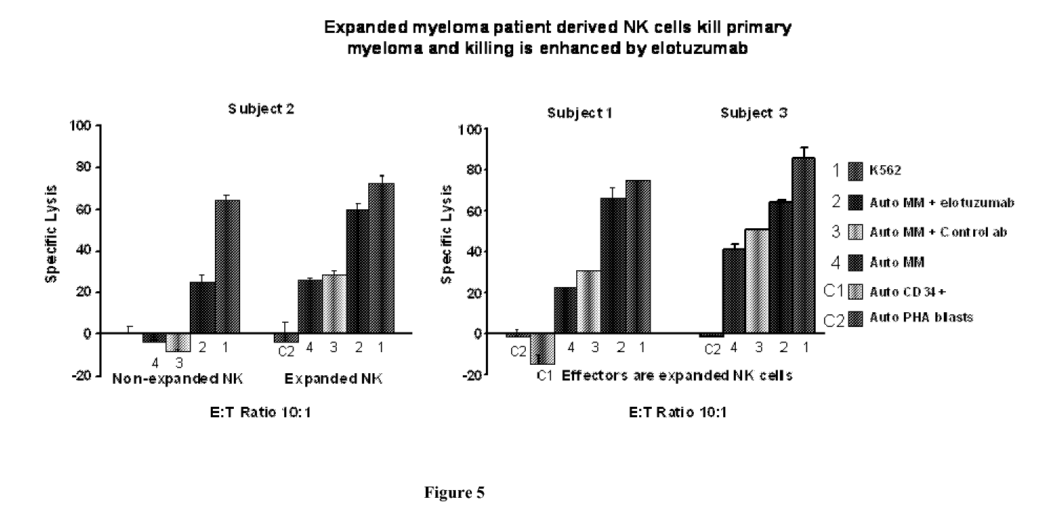

Expanded autologous NK cells killed on average about 30% of the total of cultured multiple myeloma cells from each of 3 subjects. The range of killing observed in the 3 subjects was 22-41% of cultured multiple myeloma cells. In contrast, no killing of multiple myeloma cells was observed with non-autologous NK cells that were not expanded or activated. (FIG. 5)

Pretreatment of multiple myeloma cells with the anti-CS1 antibody elotuzumab increased the expanded autologous NK cell-mediated killing of multiple myeloma cells by at least 1.7 fold over myeloma cells pretreated with an isotype control antibody or over myeloma cells that were not pretreated. The level of killing in the elotuzumab pretreatment/expanded NK cell treatment samples is comparable to the level of killing of the highly NK-kill sensitive cell line K562. In contrast, autologous PHA blasts and CD34+ stem cells were not killed.

### 7. EXAMPLE 3

### Distribution of Expanded NK Cells in the Bodies of Nod-Skid Mice

7.1 Methods

In order to determine the ability of ex vivo expanded NK cells to traffic to the bone marrow, activated NK cells were injected into the vein of NK cell depleted NOD-SCID mice, which were then sacrificed 0, 4 or 48 hours after injection. Peripheral blood, bone marrow and spleen tissue was harvested from each mouse and stained for flow cytometry. Samples were contacted with the following antibodies: anti-CD3 fluorescein isothiocyanate (FITC), anti-CD56 phycoerythrin (PE) and anti-CD45-PERCP. Antibody staining of peripheral blood, bone marrow and spleen tissue samples collected 0, 4 and 48 hours after injection was detected by flow cytometer.

7.2 Results

Activated NK cells (i.e., that express CD56, but not CD3) were detected in the bone marrow of mice at 48 hours after injection, indicating that NK cells traffic to the primary site of multiple myeloma in vivo.

### 8. EXAMPLE 4

### Treatment of Multiple Myeloma with a Combination of Elotuzumab and Autologous NK Cells

A large volume leukapheresis to collect autologous PMBCs will be performed on patients prior to administration of the combination of expanded autologous NK cells and elotuzumab. PBMCs are co-cultured for one week in stem cell growth medium (CellGenix, Freiburg, Germany), or X-VIVO serum-free media (BioWhittaker, Verviers, Belgium), which can be supplemented with fetal bovine serum from certified sources or human serum from an AB blood donor, and to which an antibiotic such as gentamycin (50 mg/l) and from 10 to 1000 IU/ml human IL-2 are added. Irradiated K562-mb15-41BBL cells (30 Gy-100 Gy) are added at a ratio of 1:10 K562-mb15-41BBL:NK cells. Cells can be cultured in flasks or in bags (e.g., Teflon (FEP) bags, Baxter Lifecell bags or VueLife bags). Cells are fed after 2 and 5 days and harvested after 7 days of culture. The cell product is then depleted of residual T-cells using the CliniMACS System (Miltenyi), and cells are then washed and resuspended in PlasmaLyte-148 (Baxter, Deerfield, Ill.) with 0.5% human serum albumin. Expansion of CD56⁺CD3⁻ NK cells is about 90-fold.

Patients will receive an intravenous infusion of elotuzumab prior to the autologous NK cell infusions. Depending on the need of the patient and at the discretion of the investigator, elutozomab can be administered at dose levels ranging from 0.5 mg/kg to 20 mg/kg.

Autologous NK cells will be transfused over approximately 8 hours by gravity. The target number of NK cells to be infused is 5×10⁵-4×10⁷ NK cells/kg. The recipient (i.e., subject) will receive standard monitoring for receiving cell products from a donor.

### 9. SPECIFIC EMBODIMENTS, CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of treating multiple myeloma comprising administering to a human patient in need thereof an effective amount of expanded and activated autologous NK cells and an effective amount of an
(a) antibody that targets myeloma cells or antigen binding fragment thereof, or an antibody-drug conjugate comprising an antibody that targets myeloma cells or antigen binding fragment conjugated to a cytotoxic agent,
(b) anti-CS1 antibody or antigen binding fragment thereof, or an anti-CS1 antibody-drug conjugate comprising anti-CS1 antibody or antigen binding fragment conjugated to a cytotoxic agent, or
(c) an effective amount of an antibody that targets the KIR protein of NK cells or antigen binding fragment thereof,
wherein the autologous NK cells have been expanded and activated by culturing in the presence of K562 cells that express 4-1BBL and IL-15 on the cell surface, and wherein the antibody that targets myeloma cells or antigen binding fragment thereof or antibody-drug conjugate elicits antibody-dependent cellular cytoxicity.

2-3. (canceled)

4. A method of treating multiple myeloma comprising administering to a human patient in need thereof (i) an effective amount of expanded and activated autologous NK cells, (ii) an effective amount of an antibody that targets the KIR protein of NK cells or antigen binding fragment thereof, and (iii) an effective amount of an antibody that targets myeloma cells or antigen binding fragment thereof, or an antibody-drug conjugate comprising an antibody that targets myeloma cells or antigen binding fragment conjugated to a cytotoxic agent, wherein the autologous NK cells have been expanded and activated by culturing in the presence of K562 cells that express 4-1BBL and IL-15 on the cell surface, and wherein the antibody that targets the KIR protein of NK cells or antigen binding fragment thereof, and the antibody that targets myeloma cells or antigen binding fragment thereof or antibody-drug conjugate elicit antibody-dependent cellular cytoxicity toward multiple myeloma cells.

5. The method of claim 1, further comprising before the administering step a step of culturing NK cells obtained from peripheral blood mononuclear cells of the patient in the presence of K562 cells that express 4-1BBL and IL-15 on the cell surface under conditions whereby the NK cells are expanded at least about 25-fold relative to the number of NK cells in the starting culture.

6. The method of claim 5, further comprising before the culturing step a step of isolating perphiperal blood mononuclear cells from the patient.

7. The method of claim 1, wherein the NK cells are cultured with from 10 to 1000 IU/ml human IL-2.

8. The method of claim 1, wherein the K562 cells are present in the autologous NK cell culture at a ratio of 1:10 K562 cells:NK cells.

9. The method of claim 1, wherein the autologous NK cells are expanded at least about 50-fold relative to the number of NK cells in the starting culture before expansion.

10-11. (canceled)

12. The method of claim (1b), wherein the anti-CS1 antibody or antigen binding fragment thereof, or the anti-CS1 antibody-drug conjugate, comprises heavy chain CDR sequences with at least 85% sequence identity to the CDR sequences of SEQ ID NOS:11, 12 and 13.

13. The method of claim (1b), wherein the anti-CS1 antibody or antigen binding fragment thereof, or the anti-CS1 antibody-drug conjugate, comprises light chain CDR sequences with at least 85% sequence identity to the CDR sequences of SEQ ID NOS:14, 15 and 16.

14. The method of claim (1b), wherein the anti-CS1 antibody or antigen binding fragment thereof, or the anti-CS1 antibody-drug conjugate, comprises a heavy chain variable region of SEQ ID NO:9 and a light chain variable region of SEQ ID NO:10.

15. The method of claim (1b), wherein the anti-CS1 antibody or antigen binding fragment thereof, or the anti-CS1 antibody-drug conjugate, competes with monoclonal antibody Luc63, as produced by the hybridoma deposited with the American Type Culture Collection (“ATCC”) and assigned accession no. PTA-5950, for binding to CS1.

16. The method of claim (1b), wherein the effective amount of the anti-CS1 antibody or antigen binding fragment thereof, or the anti-CS1 antibody-drug conjugate, is from about 0.5 mg/kg to about 20 mg/kg.

17. The method of claim 1, wherein the effective amount of autologous NK cells is from about 5×10⁵ mg/kg to about 5×10⁷ mg/kg of body weight of the subject.

18. The method of claim (1b), wherein the effective amount of the anti-CS1 antibody or antigen binding fragment thereof, or the anti-CS1 antibody-drug conjugate, is administered simultaneously with, prior to or sequentially to the administration of the effective amount of autologous NK cells.

19. (canceled)

20. The method of claim 1, wherein the antibody and the autologous NK cells are administered in separate dosage forms.

21-22. (canceled)

23. The method of claim 1, wherein the antibody and the autologous NK cells are administered with one or more additional agents.

24-28. (canceled)

29. The method of claim 1, wherein the subject has undergone stem cell transplantation prior to the administration of the effective amount of autologous NK cells and the effective amount of the antibody.

30. The method according to claim 29, wherein the stem cell transplantation is autologous stem cell transplantation.

31-39. (canceled)

40. A method of treating multiple myeloma comprising administering to a human subject in need thereof an effective amount of expanded and activated autologous NK cells and an effective amount of the anti-CS1 antibody elotuzumab, wherein the autologous NK cells have been expanded and activated by culturing in the presence of K562 cells that express 4-1BBL and IL-15 on the cell surface.

41-44. (canceled)

45. The method of claim 1, wherein the antibody that targets myeloma cells or antigen binding fragment thereof, or the antibody-drug conjugate comprising an antibody that targets myeloma cells targets a myeloma cell antigen selected from the group consisting of CD20, CD38, CD40, CD56, CD74, CD138, CD317, IGF receptor, IL-6 receptor, TRAIL receptor 1 and TRAIL receptor 2.

46-52. (canceled)
